(19) United States
(12) Patent Application Publication
(10) Pub. No.: US 2023/0395250 A1
(43) Pub. Date: Dec. 7, 2023

(54) CUSTOMIZATION, TROUBLESHOOTING, AND WIRELESS PAIRING TECHNIQUES FOR SURGICAL INSTRUMENTS

(71) Applicant: Cilag GmbH International, Zug (CH)

(72) Inventors: Christopher A. Denzinger, Cincinnati, OH (US); Monica L. Z. Rivard, Cincinnati, OH (US); Matthew D. Cowperthwait, Cincinnati, OH (US); Felix J. Bork, Schnürpflingen (DE); Risto Kojcev, Santa Clara, CA (US); Leonardo N. Rossoni, Rahway, NJ (US); Chunhui Xie, San Francisco, CA (US)

(21) Appl. No.: 18/198,036

(22) Filed: May 16, 2023

Related U.S. Application Data
(60) Provisional application No. 63/348,218, filed on Jun. 2, 2022.

(51) Int. Cl. G16H 40/63; G16H 20/40

(52) U.S. Cl. CPC G16H 40/63; G16H 20/40; A61B 34/25

(57) ABSTRACT

Systems for customizing and troubleshooting the display of surgical data and wireless pairing techniques for surgical instruments are disclosed.

### CROSS-REFERENCE TO RELATED APPLICATIONS

The present application claims the benefit of priority under 35 U.S.C. § 119(e) to U.S. Provisional Application No. 63/348,218, entitled “CUSTOMIZATION, TROUBLESHOOTING, AND WIRELESS PAIRING TECHNIQUES FOR SURGICAL INSTRUMENTS”, filed on Jun. 2, 2022, which is incorporated by reference herein in its respective entirety.

### BACKGROUND

This disclosure relates to apparatuses, systems, and methods for providing an augmented reality interactive experience during a surgical procedure. During a surgical procedure it would be desirable to provide customization to provide an updated staff view screen designed to enable customization of overlays on the primary endoscopic monitor. It also would be desirable to provide troubleshooting techniques employing a contextual workflow to help users troubleshoot system connections across equipment in the operating room (OR). Further, it would be desirable to provide wireless pairing. For example, it would be desirable to provide automatic prompting for pairing wireless devices to simplify workflow, overlaid information dynamic minimization, customize overlays on primary surgical display, and guided troubleshooting system connections.

### SUMMARY

In one general aspect, the present disclosure provides a method of automatic prompting for pairing wireless devices to simplify workflow is disclosed. The method comprising opening, by a circulating nurse, a package containing a surgical device/instrument; presenting, by the circulating nurse, the surgical device/instrument to a scrub nurse; retrieving, by the scrub nurse, the surgical device/instrument and battery from a sterile tray; removing, by the scrub nurse, a near field communication (NFC) card form the surgical device/instrument; inserting, by the scrub nurse, the battery into the surgical device/instrument; transmitting, by the surgical device/instrument, a request to pair to a surgical hub; receiving, from the surgical hub, instructions telling a user to tap the NFC card to pair or enter a 3 or 4 digit code; tapping, by the circulating nurse, the NFC card to an NFC reader on the surgical hub; pairing the surgical hub with the surgical device/instrument; and providing a confirmation screen.

In another aspect, the present disclosure provides a method of automatic prompting for pairing wireless devices to simplify workflow. The method comprising tapping a near field communication (NFC) card on a surgical device/instrument; searching, by a surgical hub, for a specific surgical device/instrument based on the media access control (MAC) address written on the NFC card; inserting, by a scrub nurse, a battery into the surgical device/instrument; pairing the surgical hub with the surgical device/instrument; and providing a confirmation screen.

In another aspect, the present disclosure provides a method of automatic prompting for pairing wireless devices to simplify workflow. The method comprising opening, by a circulating nurse, a package containing a surgical device/instrument; presenting, by the circulating nurse, the surgical device/instrument to a scrub nurse; retrieving, by the scrub nurse the surgical device/instrument and battery from a sterile tray; inserting, by the scrub nurse, the battery into the surgical device/instrument; transmitting, by the surgical device/instrument, a request to pair to a surgical hub; requesting, from the surgical hub, that the circulating nurse confirm a multi digit identification number written on the surgical device/instrument; entering, by the circulating nurse, the multi digit number written on the side of the surgical device/instrument into the surgical hub.

In another aspect, the present disclosure provides a method of dynamic minimization of overlaid information on an instrument panel display screen. The method comprising minimizing the instrument panel display screen after a predetermined time without detected interaction to a smaller panel size that shows only a subset of information or reduces scale of information.

In another aspect, the present disclosure provides a method of dynamic minimization of overlaid information on an instrument panel display screen. The method comprising removing the instrument panel display screen after a predetermined time without detected interaction.

In another aspect, the present disclosure provides a method of dynamic minimization of overlaid information on an instrument panel display screen. The method comprising changing opacity of the instrument panel display screen after a predetermined time without detected interaction.

In another aspect, the present disclosure provides a method of displaying surgical data. The method comprising displaying an overlay over a primary surgical display, the overlay providing helpful information to a surgeon or operating room (OR) staff; displaying a secondary user interface to control which overlays are shown on the primary surgical display. The secondary user interface is configured to control how the overlays are displayed by adjusting position, scale, and amount of information shown for each overlay element. The method further comprises simultaneously adjusting individual elements of overlaid information, or groups of information, through the secondary user interface. The method further comprises displaying changes instantaneously or after a delay on the primary surgical display as they are made on the secondary user interface.

In another aspect, the present disclosure provides a computer implemented surgical system for use during a surgical procedure. The computer implemented surgical system comprises a near field communication (NFC) reader and a control circuit comprising a processor and a memory. The memory stores instructions that, when executed by the processor, cause the control circuit to receive surgeon identification data indicative of a surgeon performing the surgical procedure, receive a request to wirelessly pair with a surgical device based on the surgical device powering on, and receive device identification data indicative of the surgical device. The memory stores further instructions that, when executed by the processor, cause the control circuit to wirelessly pair the surgical device to the computer implemented surgical system based on the device identification data, and retrieve surgeon preferences relating to the paired surgical device and the surgeon from a database comprising a plurality of surgeon preferences for a plurality of surgical devices. The surgeon preferences comprise an overlay configuration for at least one overlay relating to the surgical device. The memory stores further instructions that, when executed by the processor, cause the control circuit to display the at least one overlay based on the overlay configuration on a primary surgical display, wherein the overlay is displayed overtop of information on the primary surgical display during the surgical procedure.

In at least one aspect, the surgeon preferences further comprises control parameters of the paired surgical device, and wherein the memory stores further instructions that, when executed by the processor, cause the control circuit to transmit the control parameters to the surgical device.

In at least one aspect, the at least one overlay relates to data received from the surgical device during the surgical procedure, and wherein the at least one overlay is configured to provide a warning to the surgeon based on the data exceeding a predetermined threshold.

In at least one aspect, receive device identification data indicative of the surgical device comprises receiving the device identification data from a NFC card located proximal to the NFC reader, and the NFC card is stored within the surgical device prior to the surgical procedure.

In at least one aspect, the memory stores further instructions that, when executed by the processor, cause the control circuit to transmit, in response to receiving the request, instructions for wirelessly pairing with the surgical device to a display screen. In at least one aspect, the instructions for wirelessly pairing with the surgical device comprise requesting that the NFC card be placed proximal to the NFC reader.

In at least one aspect, the memory stores further instructions that, when executed by the processor, causes the control circuit to provide feedback in response to the instructions being transmitted, wherein the feedback is configured to notify a user that a user input is required.

In another aspect, the present disclosure provides a computer implemented surgical system to automatically prompt for pairing of wireless devices during a surgical procedure. The computer implemented system comprises a near field communication (NFC) reader and a control circuit comprising a processor and a memory. The memory stores instructions that, when executed by the processor, cause the control circuit to receive identification data indicative of the surgical device from a NFC card located proximal to the NFC card reader, compare the identification data to a database comprising data of a plurality of surgical devices, and determine a surgical device of the plurality of surgical devices based on the comparison. The memory stores further instructions that, when executed by the processor, cause the control circuit to wirelessly pair with the surgical device based on the data and the surgical device powering on, and transmit confirmation of the pairing to a display.

In at least one aspect, the memory stores further instructions that, when executed by the processor, cause the control circuit to receive second identification data indicative of a surgeon performing the surgical procedure, retrieve, in response to receiving the second identification data, surgeon preferences relating to the surgical device from a database comprising a plurality of surgeon preferences for surgical devices, and transmit the surgeon preferences to the surgical device.

In at least one aspect, the surgeon preferences comprise an overlay configuration for at least one overlay relating to data received from the surgical device during the surgical procedure. In at least one aspect, the memory stores further instructions that, when executed by the processor, cause the control circuit to determine an overlay configuration based on the surgeon preferences, and display the at least one overlay based on the overlay configuration on a primary surgical display. The overlay is displayed overtop of information on the primary surgical display.

In at least one aspect, the data indicative of the surgical device comprises a media access control (MAC) address, and wherein the database of a plurality of surgical devices comprises MAC addresses for a plurality of surgical devices.

In at least one aspect, the NFC card is stored within the surgical device prior to the surgical procedure.

In at least one aspect, the memory stores further instructions that, when executed by the processor, cause the control circuit to determine that the surgical device has turned on.

In another aspect, the present disclosure provides a computer implemented surgical system for use during a surgical procedure. The computer implemented surgical system comprising a control circuit comprising a processor and a memory, wherein the memory stores instructions that, when executed by the processor, cause the control circuit to receive an overlay configuration for at least one overlay from a secondary user interface. The at least one overlay comprises at least one overlay element, and the overlay configuration is based on preferences of a surgeon performing the surgical procedure. The memory stores further instructions that, when executed by the processor, cause the control circuit to display the at least one overlay based on the overlay configuration on a primary surgical display. The overlay provides information to the surgeon, and the overlay is displayed overtop of information on the primary surgical display.

In at least one aspect, the secondary user interface selects the overlay configuration from a database comprising a plurality of overlay configurations, and the selected overlay configuration is based on the surgeon.

In at least one aspect, the secondary user interface allows a user to control how the at least one overlay is displayed by adjusting position, scale, and amount of information shown for each overlay element of the at least one overlay.

In at least one aspect, the secondary user interface allows the user to update an overlay configuration and store the updated overlay configuration in a database comprising a plurality of overlay configurations.

In at least one aspect, the memory stores further instructions that, when executed by the processor, cause the control circuit to receive a second overlay configuration from the secondary user interface, and adjust simultaneously all overlay elements of the at least one overlay based on the second overlay configuration.

In at least one aspect, the at least one overlay relates to a surgical instrument, and the memory stores further instructions that, when executed by the processor, cause the control circuit to receive real-time video data of a surgical site from a camera, determine that the surgical instrument is not currently in a field of view of the camera, and adjust, automatically, the at least one overlay based on the surgical instrument leaving the field of view, wherein the adjustment minimizes the at least one overlay.

In at least one aspect, the at least one overlay relates to a surgical instrument, and wherein the memory stores further instructions that, when executed by the processor, cause the control circuit to receive data indicative of interactions of the surgical instrument, determine that the surgical instrument is inactive based on the data, and adjust, automatically, the at least one overlay based on the surgical instrument remaining inactive for a threshold period of time, wherein the adjustment minimizes the at least one overlay.

In at least one aspect, minimizing the at least one overlay comprises reducing a size of the overlay, removing the overlay, or changing an opacity of the overlay, or combinations thereof.

In at least one aspect, the secondary user interface is located outside of a sterile field and the surgical procedure is being performed within the sterile field.

### FIGURES

The various aspects described herein, both as to organization and methods of operation, together with further objects and advantages thereof, may best be understood by reference to the following description, taken in conjunction with the accompanying drawings as follows.

FIG. 1 is a block diagram of a computer-implemented interactive surgical system, according to one aspect of the present disclosure.

FIG. 2 is a surgical system being used to perform a surgical procedure in an operating room, according to one aspect of the present disclosure.

FIG. 3 is a surgical hub paired with a visualization system, a robotic system, and an intelligent instrument, according to one aspect of the present disclosure.

FIG. 4 illustrates a surgical data network comprising a modular communication hub configured to connect modular devices located in one or more operating theaters of a healthcare facility, or any room in a healthcare facility specially equipped for surgical operations, to the cloud, according to one aspect of the present disclosure.

FIG. 5 illustrates a computer-implemented interactive surgical system, according to one aspect of the present disclosure.

FIG. 6 illustrates a surgical hub comprising a plurality of modules coupled to the modular control tower, according to one aspect of the present disclosure.

FIG. 7 illustrates an augmented reality (AR) system comprising an intermediate signal combiner positioned in the communication path between an imaging module and a surgical hub display, according to one aspect of the present disclosure.

FIG. 8 illustrates an augmented reality (AR) system comprising an intermediate signal combiner positioned in the communication path between an imaging module and a surgical hub display, according to one aspect of the present disclosure.

FIG. 9 illustrates an augmented reality (AR) device worn by a surgeon to communicate data to the surgical hub, according to one aspect of the present disclosure.

FIG. 10 illustrates a system for augmenting surgical instrument information using an augmented reality display, according to one aspect of the present disclosure.

FIG. 11 illustrates a timeline of a situational awareness surgical procedure, according to one aspect of the present disclosure.

FIG. 12 illustrates a pairing workflow if a battery is first inserted into the surgical device/instrument before pairing is established, according to at least one aspect of the present disclosure.

FIG. 13 illustrates a pairing workflow if a NFC card is first tapped on the surgical device/instrument before pairing is established, according to at least one aspect of the present disclosure.

FIG. 14 illustrates a multi digit confirmation pairing workflow, according to at least one aspect of the present disclosure.

FIG. 15 illustrates instrument panel minimization, according to at least one aspect of the present disclosure.

FIG. 16 illustrates a Staff View user interface control screen that is displayed on the main laparoscopic/endoscopic surgical display, according to at least one aspect of the present disclosure.

FIG. 17 illustrates a display overlay toggle screen with a display overlay virtual switch in the “On” position, according to at least one aspect of the present disclosure.

FIG. 18 is a version of the screen shown in FIG. 17 with the switch in the “Off” position, according to at least one aspect of the present disclosure.

FIG. 19 illustrates a presets dropdown screen, according to at least one aspect of the present disclosure.

FIG. 20 is a version of the screen shown in FIG. 19 with a new preset dropdown screen displayed after selecting the preset, according to at least one aspect of the present disclosure.

FIG. 21 illustrates a screen with an overlay size selection screen with the overlay size set to small, according to at least one aspect of the present disclosure.

FIG. 22 is a version of the screen shown in FIG. 21 with the overlay size set to large, according to at least one aspect of the present disclosure.

FIG. 23 illustrates a screen with a top level switch and an edit information screen, according to at least one aspect of the present disclosure.

FIG. 24 is a version of the screen shown in FIG. 23 with the top level switch switched off, according to at least one aspect of the present disclosure.

FIG. 25 illustrates a customized edit panel, which is opened by tapping on the text of the edit information screen shown in FIG. 23, according to at least one aspect of the present disclosure.

FIG. 26 is a version of the screen shown in FIG. 25 displayed by tapping on the Edit Case Information panel to open the Edit case Information subpanel, according to at least one aspect of the present disclosure.

FIG. 27 illustrates a screen with a customizable panel, according to at least one aspect of the present disclosure.

FIG. 28 is a version of the screen shown in FIG. 28 where the content displayed on the screen dynamically collapses based on the customized selection, according to at least one aspect of the present disclosure.

FIG. 29 illustrates an information panel screen, according to at least one aspect of the present disclosure.

FIG. 30 is a version of the screen shown in FIG. 29 with the information popup moved along an edge of the screen to the next item, according to at least one aspect of the present disclosure.

FIG. 31 illustrates a screen with an overlaid system connection banner, which provides an overview of the connection status, according to at least one aspect of the present disclosure.

FIG. 32 is version of the screen shown in FIG. 31 with the checkmark icons indicating a successful connection, according to at least one aspect of the present disclosure.

FIG. 33 illustrates a troubleshooting window, according to at least one aspect of the present disclosure.

FIG. 34 displays a more detailed troubleshooting instruction in a pop-up window shown in screen that overlays (partially or completely) the primary interface, according to at least one aspect of the present disclosure.

FIGS. 35A and 35B illustrate a troubleshooting flow diagram, according to at least one aspect of the present disclosure.

Corresponding reference characters indicate corresponding parts throughout the several views. The exemplifications set out herein illustrate various disclosed embodiments, in one form, and such exemplifications are not to be construed as limiting the scope thereof in any manner.

### DESCRIPTION

Applicant of the present application owns the following U.S. Patent Application, the disclosure of which is herein incorporated by reference in its entirety:
U.S. patent application Ser. No. 17/688,589, titled METHOD FOR INTRAOPERATIVE DISPLAY FOR SURGICAL SYSTEMS, filed Mar. 7, 2022, now U.S. Patent Publication No. US-2022-0331047-A1.

Applicant of the present application owns the following U.S. Patent Applications, the disclosures of each are herein incorporated by reference in their entireties:
U.S. patent application Ser. No. 16/209,423, titled METHOD OF COMPRESSING TISSUE WITHIN A STAPLING DEVICE AND SIMULTANEOUSLY DISPLAYING THE LOCATION OF THE TISSUE WITHIN THE JAWS, now U.S. Patent Publication No. US-2019-0200981-A1; and
U.S. patent application Ser. No. 16/209,453, titled METHOD FOR CONTROLLING SMART ENERGY DEVICES, now U.S. Pat. No. 11,589,888.

Before explaining various aspects of surgical devices and generators in detail, it should be noted that the illustrative examples are not limited in application or use to the details of construction and arrangement of parts illustrated in the accompanying drawings and description. The illustrative examples may be implemented or incorporated in other aspects, variations and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects and/or examples.

Various aspects are directed to onscreen displays for surgical systems for a variety of energy and surgical stapler based medical devices. Energy based medical devices include, without limitation, radio-frequency (RF) based monopolar and bipolar electrosurgical instruments, ultrasonic surgical instruments, combination RF electrosurgical and ultrasonic instruments, combination RF electrosurgical and mechanical staplers, among others. Surgical stapler devices include combined surgical staplers with electrosurgical and/or ultrasonic devices. Aspects of the ultrasonic surgical devices can be configured for transecting and/or coagulating tissue during surgical procedures, for example. Aspects of the electrosurgical devices can be configured for transecting, coagulating, sealing, welding and/or desiccating tissue during surgical procedures, for example. Aspects of the surgical stapler devices can be configured for transecting and stapling tissue during surgical procedures and in some aspects, the surgical stapler devices may be configured to delivery RF energy to the tissue during surgical procedures. Electrosurgical devices are configured to deliver therapeutic and/or nontherapeutic RF energy to the tissue. Elements of surgical staplers, electrosurgical, and ultrasonic devices may be used in combination in a single surgical instrument.

In various aspects, the present disclosure provides onscreen displays of real time information to the OR team during a surgical procedure. In accordance with various aspects of the present disclosure, many new and unique onscreen displays are provided to display onscreen a variety of visual information feedback to the OR team. According to the present disclosure, visual information may comprise one or more than one of various visual media with or without sound. Generally, visual information comprises still photography, motion picture photography, video or audio recording, graphic arts, visual aids, models, display, visual presentation services, and the support processes. The visual information can be communicated on any number of display options such as the primary OR screen, the energy or surgical stapler device itself, a tablet, augmented reality glasses, among others, for example.

In various aspects, the present disclosure provides a large list of potential options to communicate visual information in real time to the OR team, without overwhelming the OR team with too much visual information. For example, in various aspects, the present disclosure provides onscreen displays of visual information to enable the surgeon, or other members of the OR team, to selectively activate onscreen displays such as icons surrounding the screen option to manage a wealth of visual information. One or a combination of factors can be used to determine the active display, these may include energy based (e.g., electrosurgical, ultrasonic) or mechanical based (e.g., staplers) surgical devices in use, the estimated risk associated with a given display, the experience level of the surgeon and the surgeons' choice among other things. In other aspects, the visual information may comprises rich data overlaid or superimposed into the surgical field of view to manage the visual information. In various aspects described herein below, the visual information comprises superimposed imagery that requires video analysis and tracking to properly overlay the data. Visual information data communicated in this manner, as opposed to static icons, may provide additional useful visual information in a more concise and easy to understand way to the OR team.

In various aspects, the present disclosure provides techniques for selectively activating onscreen displays such as icons surrounding the screen to manage visual information during a surgical procedure. In other aspects, the present disclosure provides techniques for determining the active display using one or a combination of factors. In various aspects, the techniques according to the resent disclosure may comprise selecting the energy based or mechanical based surgical device in use as the active display, estimating risk associated with a given display, utilizing the experience level of the surgeon or OR team making the selection, among other things.

In other aspects, the techniques according to the present disclosure may comprise overlaying or superimposing rich data onto the surgical field of view to manage the visual information. A number of the display arrangements described by the present disclosure involve overlaying various visual representations of surgical data onto a livestream of a surgical field. As used herein the term overlay comprises a translucent overlay, a partial overlay, and/or a moving overlay. Graphical overlays may be in the form of a transparent graphic, semitransparent graphic, or opaque graphic, or a combination of transparent, semitransparent, and opaque elements or effects. Moreover, the overlay can be positioned on, or at least partially on, or near an object in the surgical field such as, for example, an end effector and/or a critical surgical structure. Certain display arrangements may comprise a change in one or more display elements of an overlay including a change in color, size, shape, display time, display location, display frequency, highlighting, or a combination thereof, based on changes in display priority values. The graphical overlays are rendered on top of the active display monitor to convey important information quickly and efficiently to the OR team.

In other aspects, the techniques according to the present disclosure may comprise superimposing imagery that requires analyzing video and tracking for properly overlaying the visual information data. In other aspects, the techniques according to the present disclosure may comprise communicating rich visual information, as opposed to simple static icons, to provide additional visual information to the OR team in a more concise and easy to understand manner. In other aspects, the visual overlays may be used in combination with audible and/or somatosensory overlays such as thermal, chemical, and mechanical devices, and combinations thereof.

The following description is directed generally to apparatuses, systems, and methods that provide an augmented reality (AR) interactive experience during a surgical procedure. In this context, images of a surgical field and surgical instruments and other objects appearing in the surgical field are enhanced by overlaying computer-generated visual, auditory, haptic, somatosensory, olfactory, or other sensory information onto the real world images of the surgical field, instruments, and/or other objects appearing in the surgical field. The images may be streamed in real time or may be still images. Augmented reality is a technology for rendering and displaying virtual or “augmented” virtual objects, data, or visual effects overlaid on a real environment. The real environment may include a surgical field. The virtual objects overlaid on the real environment may be represented as anchored or in a set position relative to one or more aspects of the real environment. In a non-limiting example, if a real world object exits the real environment field of view, a virtual object anchored to the real world object would also exit the augmented reality field of view.

A number of the display arrangements described by the present disclosure involve overlaying various visual representations of surgical data onto a livestream of a surgical field. As used herein the term overlaying comprises a translucent overlay, a partial overlay, and/or a moving overlay. Moreover, the overlay can be positioned on, or at least partially on, or near an object in the surgical field such as, for example, an end effector and/or a critical surgical structure. Certain display arrangements may comprise a change in one or more display elements of an overlay including a change in color, size, shape, display time, display location, display frequency, highlighting, or a combination thereof, based on changes in display priority values.

As described herein AR is an enhanced version of the real physical world that is achieved through the use of digital visual elements, sound, or other sensory stimuli delivered via technology. Virtual Reality (VR) is a computer-generated environment with scenes and objects that appear to be real, making the user feel they are immersed in their surroundings. This environment is perceived through a device known as a Virtual Reality headset or helmet. Mixed reality (MR) and AR are both considered immersive technologies, but they aren't the same. MR is an extension of Mixed reality that allows real and virtual elements to interact in an environment. While AR adds digital elements to a live view often by using a camera, an MR experience combines elements of both AR and VR, where real-world and digital objects interact.

In an AR environment, one or more computer-generated virtual objects may be displayed along with one or more real (i.e., so-called “real world”) elements. For example, a real-time image or video of a surrounding environment may be shown on a computer screen display with one or more overlaying virtual objects. Such virtual objects may provide complementary information relating to the environment or generally enhance a user's perception and engagement with the environment. Conversely, the real-time image or video of the surrounding environment may additionally or alternatively enhance a user's engagement with the virtual objects shown on the display.

The apparatuses, systems, and methods in the context of this disclosure enhance images received from one or more imaging devices during a surgical procedure. The imaging devices may include a variety of scopes used during non-invasive and minimally invasive surgical procedures, an AR device, and/or a camera to provide images during open surgical procedures. The images may be streamed in real time or may be still images. The apparatuses, systems, and methods provide an augmented reality interactive experience by enhancing images of the real world surgical environment by overlaying virtual objects or representations of data and/or real objects onto the real surgical environment. The augmented reality experience may be viewed on a display and/or an AR device that allows a user to view the overlaid virtual objects onto the real world surgical environment. The display may be located in the operating room or remote from the operating room. AR devices are worn on the head of the surgeon or other operating room personnel and typically include two stereo-display lenses or screens, including one for each eye of the user. Natural light is permitted to pass through the two transparent or semi-transparent display lenses such that aspects of the real environment are visible while also projecting light to make virtual objects visible to the user of the AR device.

Two or more displays and AR devices may be used in a coordinated manner, for example with a first display or AR device controlling one or more additional displays or AR devices in a system with defined roles. For example, when activating a display or an AR device, a user may select a role (e.g., surgeon, surgical assistant, nurse, etc., during a surgical procedure) and the display or the AR device may display information relevant to that role. For example, a surgical assistant may have a virtual representation of an instrument displayed that the surgeon needs to perform for a next step of a surgical procedure. A surgeon's focus on the current step may see different information displayed than the surgical assistant.

Although there are many known onscreen displays and alerts, this disclosure provides many new and unique augmented reality interactive experiences during a surgical procedure. Such augmented reality interactive experiences include visual, auditory, haptic, somatosensory, olfactory, or other sensory feedback information to the surgical team inside or outside the operating room. The virtual feedback information overlaid onto the real world surgical environment may be provided to an operating room (OR) team, including personnel inside the OR including, without limitation, the operating surgeon, assistants to the surgeon, a scrub person, an anesthesiologist and a circulating nurse, among others, for example. The virtual feedback information can be communicated on any number of display options such as a primary OR screen display, an AR device, the energy or surgical stapler instrument, a tablet, augmented reality glasses, a surgical device, and/or etc.

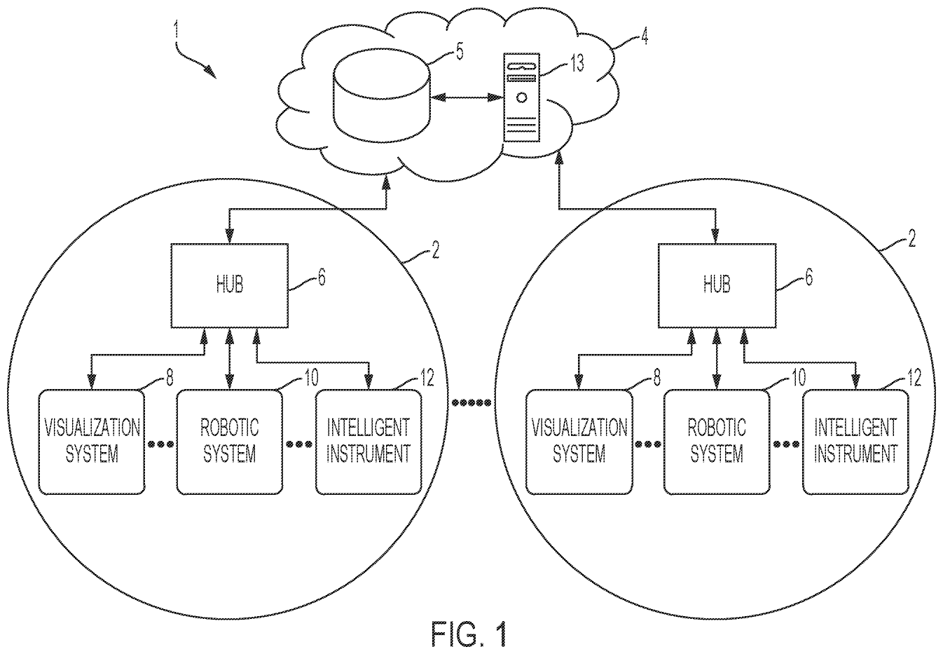

FIG. 1 depicts a computer-implemented interactive surgical system 1 that includes one or more surgical systems 2 and a cloud-based system 4. The cloud-based system 4 may include a remote server 13 coupled to a storage device 5. Each surgical system 2 includes at least one surgical hub 6 in communication with the cloud 4. For example, the surgical system 2 may include a visualization system 8, a robotic system 10, and handheld intelligent surgical instruments 12, each configured to communicate with one another and/or the hub 6. In some aspects, a surgical system 2 may include an M number of hubs 6, an N number of visualization systems 8, an O number of robotic systems 10, and a P number of handheld intelligent surgical instruments 12, where M, N, O, and P are integers greater than or equal to one. The computer-implemented interactive surgical system 1 may be configured to provide an augmented reality interactive experience during a surgical procedure as described herein.

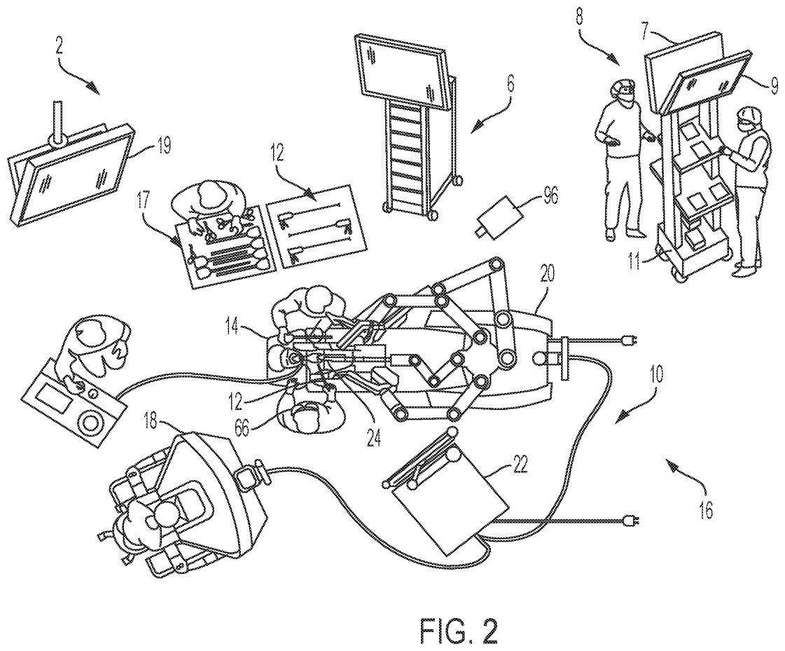

FIG. 2 depicts an example of a surgical system 2 to perform a surgical procedure on a patient lying down on an operating table 14 in a surgical operating room 16. A robotic system 10 is used in the surgical procedure as a part of the surgical system 2. The robotic system 10 includes a surgeon's console 18, a patient side cart 20 (surgical robot), and a surgical robotic hub 22. The patient side cart 20 can manipulate at least one removably coupled surgical tool 17 through a minimally invasive incision in the body of the patient while the surgeon views the surgical site through the surgeon's console 18 or an augmented reality (AR) device 66 worn by the surgeon. An image (e.g., still or live streamed in real time) of the surgical site during a minimally invasive procedure can be obtained by a medical imaging device 24. The patient side cart 20 can manipulate the imaging device 24 to orient the imaging device 24. An image of an open surgical procedure can be obtained by a medical imaging device 96. The robotic hub 22 processes the images of the surgical site for subsequent display on the surgeon's console 18 or the AR device 66 worn by the surgeon, or other person in the surgical operating room 16.

The optical components of the imaging device 24, 96 or AR device 66 may include one or more illumination sources and/or one or more lenses. The one or more illumination sources may be directed to illuminate portions of the surgical field. One or more image sensors may receive light reflected or refracted from tissue and instruments in the surgical field.

In various aspects, the imaging device 24 is configured for use in a minimally invasive surgical procedure. Examples of imaging devices suitable for use with this disclosure include, but not limited to, an arthroscope, angioscope, bronchoscope, choledochoscope, colonoscope, cytoscope, duodenoscope, enteroscope, esophagogastro-duodenoscope (gastroscope), endoscope, laryngoscope, nasopharyngo-neproscope, sigmoidoscope, thoracoscope, and ureteroscope. In various aspects, the imaging device 96 is configured for use in an open (invasive) surgical procedure.

In various aspects, the visualization system 8 includes one or more imaging sensors, one or more image-processing units, one or more storage arrays, and one or more displays that are strategically arranged with respect to the sterile field. In one aspect, the visualization system 8 includes an interface for HL7, PACS, and EMR. In one aspect, the imaging device 24 may employ multi-spectrum monitoring to discriminate topography and underlying structures. A multi-spectral image captures image data within specific wavelength ranges in the electromagnetic spectrum. Wavelengths are separated by filters or instruments sensitive to particular wavelengths, including light from frequencies beyond the visible light range, e.g., IR and ultraviolet. Spectral imaging can extract information not visible to the human eye. Multi-spectrum monitoring can relocate a surgical field after a surgical task is completed to perform tests on the treated tissue.

FIG. 2 depicts a primary display 19 positioned in the sterile field to be visible to an operator at the operating table 14. A visualization tower 11 is positioned outside the sterile field and includes a first non-sterile display 7 and a second non-sterile display 9, which face away from each other. The visualization system 8, guided by the hub 6, is configured to utilize the displays 7, 9, 19 to coordinate information flow to operators inside and outside the sterile field. For example, the hub 6 may cause the visualization system 8 to display AR images of the surgical site, as recorded by an imaging device 24, 96 on a non-sterile display 7, 9, or through the AR device 66, while maintaining a live feed of the surgical site on the primary display 19 or the AR device 66. The non-sterile display 7, 9 can permit a non-sterile operator to perform a diagnostic step relevant to the surgical procedure, for example.

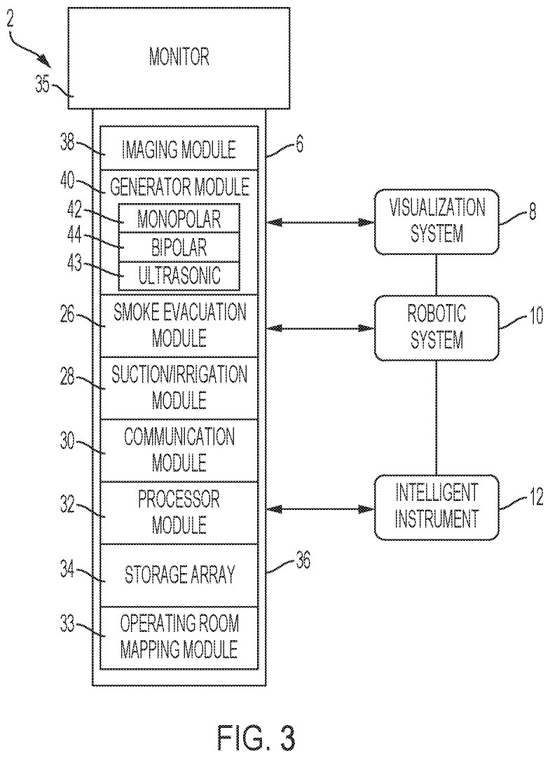

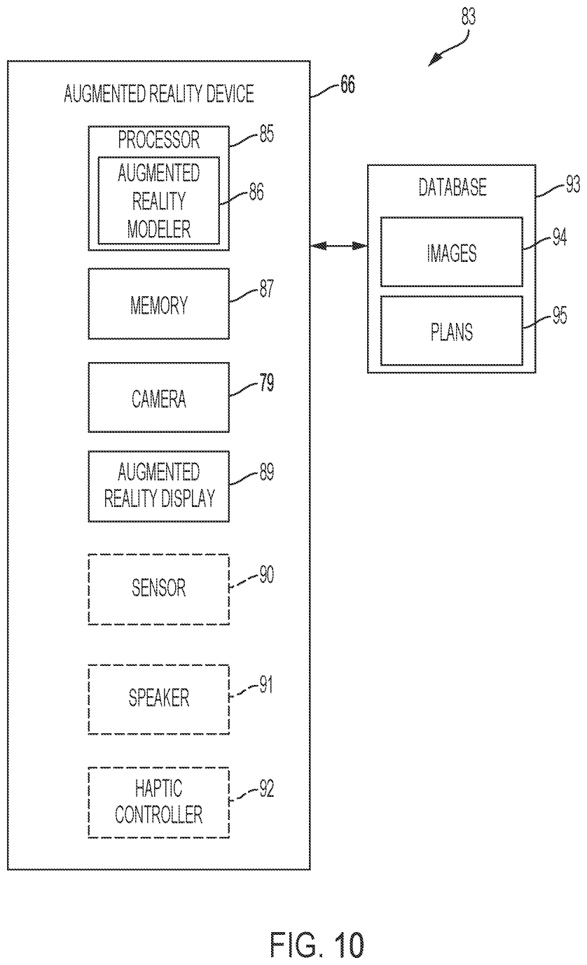

FIG. 3 depicts a hub 6 in communication with a visualization system 8, a robotic system 10, and a handheld intelligent surgical instrument 12. The hub 6 includes a hub display an imaging module 38, a generator module 40, a communication module 30, a processor module 32, a storage array 34, and an operating room mapping module 33. The hub 6 further includes a smoke evacuation module 26 and/or a suction/irrigation module 28. In various aspects, the imaging module 38 comprises an AR device 66 and the processor module 32 comprises an integrated video processor and an augmented reality modeler (e.g., as shown in FIG. 10). A modular light source may be adapted for use with various imaging devices. In various examples, multiple imaging devices may be placed at different positions in the surgical field to provide multiple views (e.g., non-invasive, minimally invasive, invasive or open surgical procedures). The imaging module 38 can be configured to switch between the imaging devices to provide an optimal view. In various aspects, the imaging module 38 can be configured to integrate the images from the different imaging devices and provide an augmented reality interactive experience during a surgical procedure as described herein.

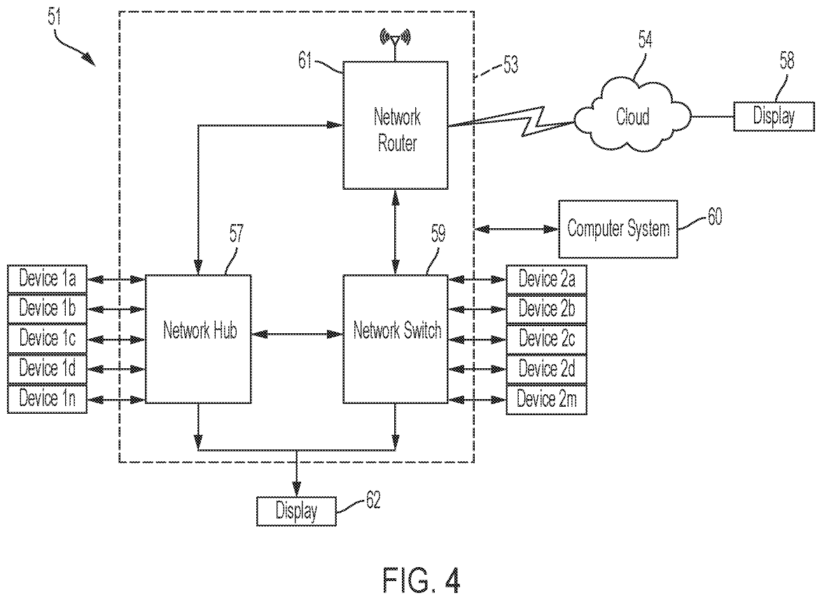

FIG. 4 shows a surgical data network 51 comprising a modular communication hub 53 configured to connect modular devices located in one or more operating theaters/rooms of a healthcare facility to a cloud-based system. The cloud 54 may include a remote server 63 (FIG. coupled to a storage device 55. The modular communication hub 53 comprises a network hub 57 and/or a network switch 59 in communication with a network router 61. The modular communication hub 53 is coupled to a local computer system 60 to process data. Modular devices la-1n in the operating theater may be coupled to the modular communication hub 53. The network hub 57 and/or the network switch 59 may be coupled to a network router 61 to connect the devices 1a-1n to the cloud 54 or the local computer system 60. Data associated with the devices 1a-1n may be transferred to cloud-based computers via the router for remote data processing and manipulation. The operating theater devices 1a-1n may be connected to the modular communication hub 53 over a wired channel or a wireless channel. The surgical data network 51 environment may be employed to provide an augmented reality interactive experience during a surgical procedure as described herein and in particular providing augmented images if the surgical field to one or more than one remote display 58.

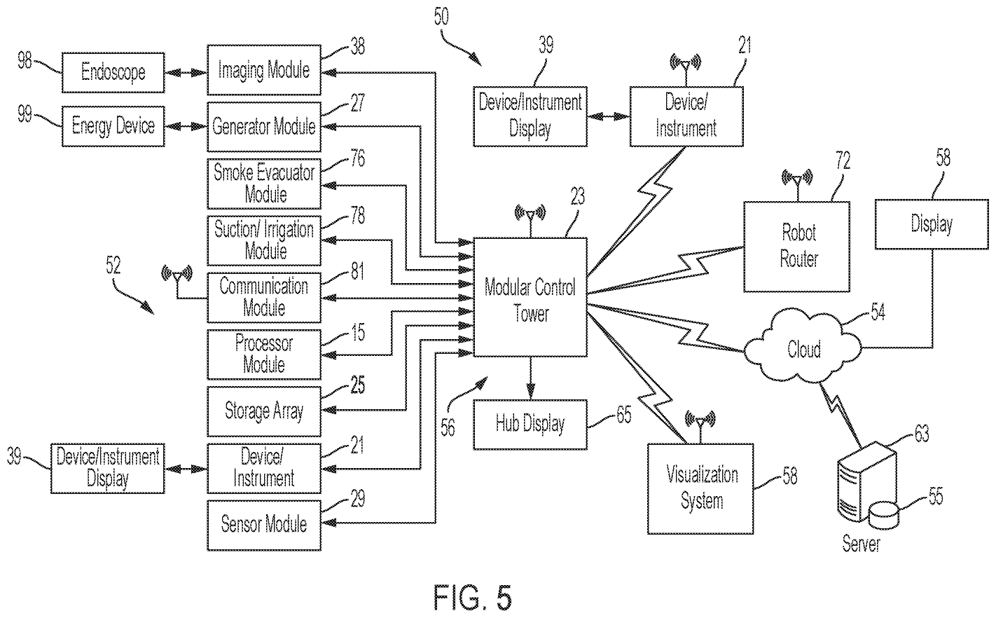

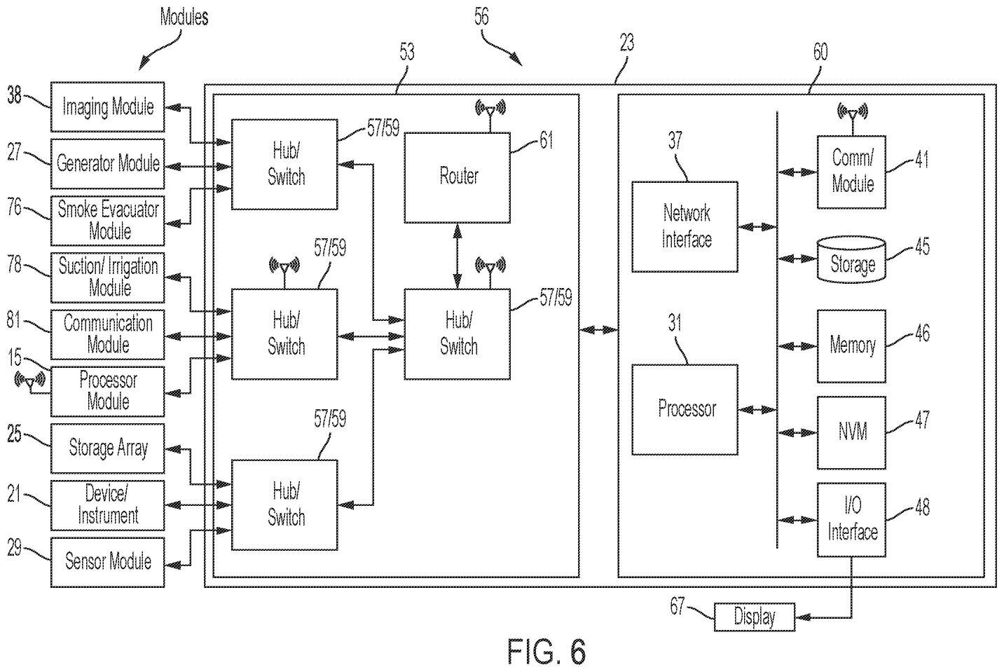

FIG. 5 illustrates a computer-implemented interactive surgical system 50. The computer-implemented interactive surgical system 50 is similar in many respects to the computer-implemented interactive surgical system 1. The computer-implemented interactive surgical system 50 includes one or more surgical systems 52, which are similar in many respects to the surgical systems 2. Each surgical system 52 includes at least one surgical hub 56 in communication with a cloud 54 that may include a remote server 63. In one aspect, the computer-implemented interactive surgical system 50 comprises a modular control tower 23 connected to multiple operating theater devices such as, for example, intelligent surgical instruments, robots, and other computerized devices located in the operating theater. As shown in FIG. 6, the modular control tower 23 comprises a modular communication hub 53 coupled to a computer system 60.

Back to FIG. 5, the modular control tower 23 is coupled to an imaging module 38 that is coupled to an endoscope 98, a generator module 27 that is coupled to an energy device 99, a smoke evacuator module 76, a suction/irrigation module 78, a communication module 81, a processor module 15, a storage array 25, a smart device/instrument 21 optionally coupled to a display 39, and a sensor module 29. The operating theater devices are coupled to cloud computing resources such as server 63, data storage 55, and displays 58 via the modular control tower 23. A robot hub 72 also may be connected to the modular control tower 23 and to the servers 63, data storage 55, and displays 58. The devices/instruments 21, visualization systems 58, among others, may be coupled to the modular control tower 23 via wired or wireless communication standards or protocols, as described herein. The modular control tower 23 may be coupled to a hub display 65 (e.g., monitor, screen) to display augmented images received comprising overlaid virtual objects on the real surgical field received from the imaging module 38, device/instrument display 39, and/or other visualization systems 58. The hub display also may display data received from devices connected to the modular control tower 23 in conjunction with images and overlaid images.

FIG. 6 illustrates a surgical hub 56 comprising a plurality of modules coupled to the modular control tower 23. The modular control tower 23 comprises a modular communication hub 53, e.g., a network connectivity device, and a computer system 60 to provide local processing, visualization, and imaging of augmented surgical information, for example. The modular communication hub 53 may be connected in a tiered configuration to expand the number of modules (e.g., devices) that may be connected to the modular communication hub 53 and transfer data associated with the modules to the computer system 60, cloud computing resources, or both. Each of the network hubs/switches 57, 59 in the modular communication hub 53 may include three downstream ports and one upstream port. The upstream network hub/switch 57, 59 is connected to a processor 31 to provide a communication connection to the cloud computing resources and a local display 67. Communication to the cloud 54 may be made either through a wired or a wireless communication channel.

The computer system 60 comprises a processor 31 and a network interface 37. The processor 31 is coupled to a communication module 41, storage 45, memory 46, non-volatile memory 47, and input/output interface 48 via a system bus. The system bus can be any of several types of bus structure(s) including the memory bus or memory controller, a peripheral bus or external bus, and/or a local bus using any variety of available bus architectures.

The processor 31 comprises an augmented reality modeler (e.g., as shown in FIG. and may be implemented as a single-core or multicore processor such as those known under the trade name ARM Cortex by Texas Instruments. In one aspect, the processor may be an LM4F230H5QR ARM Cortex-M4F Processor Core, available from Texas Instruments, for example, comprising an on-chip memory of 256 KB single-cycle flash memory, or other non-volatile memory, up to 40 MHz, a prefetch buffer to improve performance above 40 MHz, a 32 KB single-cycle serial random access memory (SRAM), an internal read-only memory (ROM) loaded with StellarisWare® software, a 2 KB electrically erasable programmable read-only memory (EEPROM), and/or one or more pulse width modulation (PWM) modules, one or more quadrature encoder inputs (QEI) analogs, one or more 12-bit analog-to-digital converters (ADCs) with 12 analog input channels, details of which are available for the product datasheet.

The system memory includes volatile memory and non-volatile memory. The basic input/output system (BIOS), containing the basic routines to transfer information between elements within the computer system, such as during start-up, is stored in non-volatile memory. For example, the non-volatile memory can include ROM, programmable ROM (PROM), electrically programmable ROM (EPROM), EEPROM, or flash memory. Volatile memory includes random-access memory (RAM), which acts as external cache memory. Moreover, RAM is available in many forms such as SRAM, dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM).

The computer system 60 also includes removable/non-removable, volatile/non-volatile computer storage media, such as for example disk storage. The disk storage includes, but is not limited to, devices like a magnetic disk drive, floppy disk drive, tape drive, Jaz drive, Zip drive, LS-60 drive, flash memory card, or memory stick. In addition, the disk storage can include storage media separately or in combination with other storage media including, but not limited to, an optical disc drive such as a compact disc ROM device (CD-ROM), compact disc recordable drive (CD-R Drive), compact disc rewritable drive (CD-RW Drive), or a digital versatile disc ROM drive (DVD-ROM). To facilitate the connection of the disk storage devices to the system bus, a removable or non-removable interface may be employed.

In various aspects, the computer system 60 of FIG. 6, the imaging module 38 and/or visualization system 58, and/or the processor module 15 of FIGS. 4-6, may comprise an image processor, image-processing engine, graphics processing unit (GPU), media processor, or any specialized digital signal processor (DSP) used for the processing of digital images. The image processor may employ parallel computing with single instruction, multiple data (SIMD) or multiple instruction, multiple data (MIMD) technologies to increase speed and efficiency. The digital image-processing engine can perform a range of tasks. The image processor may be a system on a chip with multicore processor architecture.

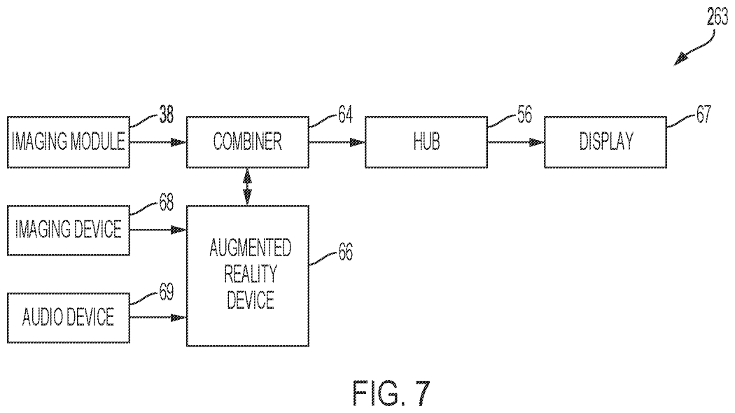

FIG. 7 illustrates an augmented reality system 263 comprising an intermediate signal combiner 64 positioned in the communication path between an imaging module 38 and a surgical hub display 67. The signal combiner 64 combines audio and/or image data received from an imaging module 38 and/or an AR device 66. The surgical hub 56 receives the combined data from the combiner 64 and overlays the data provided to the display 67, where the overlaid data is displayed. The imaging device 68 may be a digital video camera and the audio device 69 may be a microphone. The signal combiner 64 may comprise a wireless heads-up display adapter to couple to the AR device 66 placed into the communication path of the display 67 to a console allowing the surgical hub 56 to overlay data on the display 67.

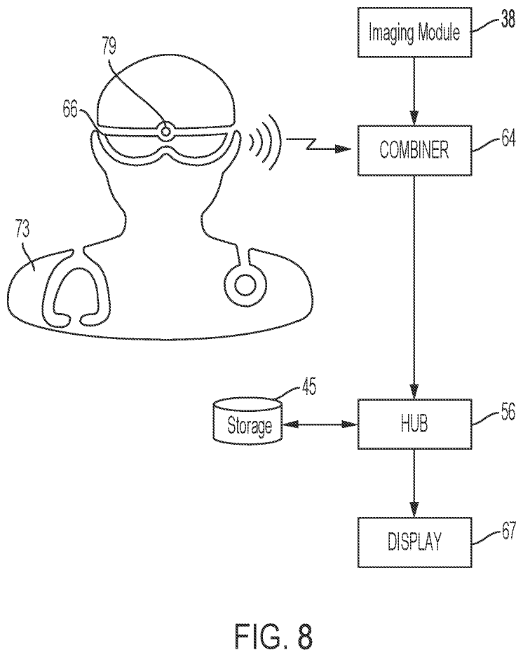

FIG. 8 illustrates an augmented reality (AR) system comprising an intermediate signal combiner positioned in the communication path between an imaging module and a surgical hub display. FIG. 8 illustrates an AR device 66 worn by a surgeon 73 to communicate data to the surgical hub 56. Peripheral information of the AR device 66 does not include active video. Rather, the peripheral information includes only device settings, or signals that do not have same demands of refresh rates. Interaction may augment the surgeon's 73 information based on linkage with preoperative computerized tomography (CT) or other data linked in the surgical hub 56. The AR device 66 can identify structure, for example allowing a surgeon to ask whether an instrument is touching a nerve, vessel, or adhesion. The AR device 66 may include pre-operative scan data, an optical view, tissue interrogation properties acquired throughout a surgical procedure, and/or processing in the surgical hub 56 used to provide information, for example an answer to a question. The surgeon 73 can dictate notes to the AR device 66 to be saved with patient data in the hub storage 45 for later use in a report or in a follow up.

The AR device 66 worn by the surgeon 73 links to the surgical hub 56 with audio and visual information to avoid the need for overlays, and allows customization of displayed information around the periphery of view. The AR device 66 provides signals from devices (e.g., instruments), answers queries about device settings, and/or positional information linked with video data to identify quadrant or position. The AR device 66 has audio control and audio feedback from the AR device 66. The AR device 66 is able to interact with other systems in the operating theater and have feedback and interaction available wherever the surgeon 73 is viewing. For example, the AR device 66 may receive voice or gesture initiated commands and queries from a surgeon, and the AR device 66 may provide feedback in the form of one or more modalities including audio, visual, or haptic touch.

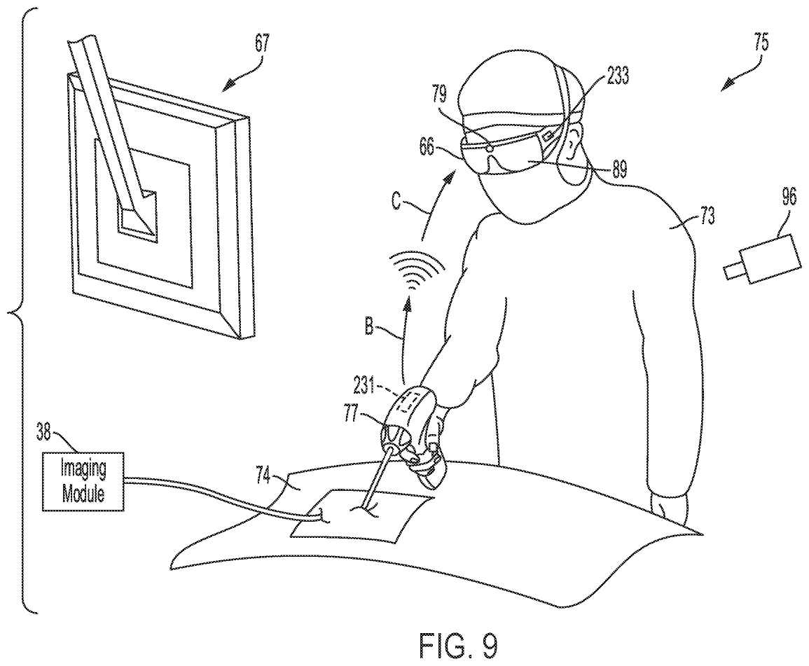

FIG. 9 illustrates a surgeon 73 wearing an AR device 66, a patient 74, and may include a camera 96 in an operating room 75. The AR device 66 worn by the surgeon 73 may be used to present to the surgeon 73 a virtual object overlaid on a real time image of the surgical field through augmented reality display 89 or through the hub connected display 67. The real time image may include a portion of a surgical instrument 77. The virtual object may not be visible to others within the operating room 75 (e.g., surgical assistant or nurse), though they also may wear AR devices 66. Even if another person is viewing the operating room 75 with an AR device 66, the person may not be able to see the virtual object or may be able to see the virtual object in a shared augmented reality with the surgeon 73, or may be able to see a modified version of the virtual object (e.g., according to customizations unique to the surgeon 73) or may see different virtual objects.

A virtual object and/or data may be configured to appear on a portion of a surgical instrument 77 or in a surgical field of view captured by an imaging module 38, an imaging device 68 during minimally invasive surgical procedures, and/or the camera 96 view during open surgical procedures. In the illustrated example, the imaging module 38 is a laparoscopic camera that provides a live feed of a surgical area during a minimally invasive surgical procedure. An AR system may present virtual objects that are fixed to a real object without regard to a perspective of a viewer or viewers of the AR system (e.g., the surgeon 73). For example, a virtual object may be visible to a viewer of the AR system inside the operating room and not visible to a viewer of the AR system outside the operating room 75. The virtual object may be displayed to the viewer outside the operating room 75 when the viewer enters the operating room 75. The augmented image may be displayed on the surgical hub display 67 or the augmented reality display 89.

The AR device 66 may include one or more screens or lens, such as a single screen or two screens (e.g., one per eye of a user). The screens may allow light to pass through the screens such that aspects of the real environment are visible while displaying the virtual object. The virtual object may be made visible to the surgeon 73 by projecting light. A virtual object may appear to have a degree of transparency or may be opaque (i.e., blocking aspects of the real environment).

An AR system may be viewable to one or more viewers, and may include differences among views available for the one or more viewers while retaining some aspects as universal among the views. For example, a heads-up display may change between two views while virtual objects and/or data may be fixed to a real object or area in both views. Aspects such as a color of an object, lighting, or other changes may be made among the views without changing a fixed position of at least one virtual object.

A user may see a virtual object and/or data presented in an AR system as opaque or as including some level of transparency. In an example, the user may interact with the virtual object, such as by moving the virtual object from a first position to a second position. For example, the user may move an object with his or her hand. This may be done in the AR system virtually by determining that the hand has moved into a position coincident or adjacent to the object (e.g., using one or more cameras, which may be mounted on the AR device 66, such as AR device camera 79 or separate imaging device 96, and which may be static or may be controlled to move), and causing the object to move in response. Virtual aspects may include virtual representations of real world objects or may include visual effects, such as lighting effects, etc. The AR system may include rules to govern the behavior of virtual objects, such as subjecting a virtual object to gravity or friction, or may include other predefined rules that defy real world physical constraints (e.g., floating objects, perpetual motion, etc.). The AR device 66 may include a camera 79 on the AR device 66 (not to be confused with the camera 96, separate from the AR device 66). The AR device camera 79 or the camera 96 may include an infrared camera, an infrared filter, a visible light filter, a plurality of cameras, a depth camera, etc. The AR device 66 may project virtual items over a representation of a real environment, which may be viewed by a user.

The AR device 66 may be used in the operating room 75 during a surgical procedure, for example performed by the surgeon 73 on the patient 74. The AR device 66 may project or display virtual objects, such as a virtual object during the surgical procedure to augment the surgeon's vision. The surgeon 73 may view a virtual object using the AR device 66, a remote controller for the AR device 66, or may interact with a virtual object, for example, using a hand to “interact” with a virtual object or a gesture recognized by the camera 79 of the AR device 66. A virtual object may augment a surgical tool such as the surgical instrument 77. For example, the virtual object may appear (to the surgeon 73 viewing the virtual object through the AR device 66) to be coupled with or remain a fixed distance from the surgical instrument 77. In another example, the virtual object may be used to guide the surgical instrument 77, and may appear to be fixed to the patient 74. In certain examples, a virtual object may react to movements of other virtual or real-world objects in the surgical field. For example, the virtual object may be altered when a surgeon is manipulating a surgical instrument in proximity to the virtual object.

The augmented reality display system imaging device 38 captures a real image of a surgical area during a surgical procedure. An augmented reality display 89, 67 presents an overlay of an operational aspect of the surgical instrument 77 onto the real image of the surgical area. The surgical instrument 77 includes communications circuitry 231 to communicate operational aspects and functional data from the surgical instrument 77 to the AR device 66 via communication communications circuitry 233 on the AR device 66. Although the surgical instrument 77 and the AR device 66 are shown in RF wireless communication between circuits 231, 233 as indicated by arrows B, C, other communication techniques may employed (e.g., wired, ultrasonic, infrared, etc.). The overlay is related to the operational aspect of the surgical instrument 77 being actively visualized. The overlay combines aspects of tissue interaction in the surgical area with functional data from the surgical instrument 77. A processor portion of the AR device 66 is configured to receive the operational aspects and functional data from the surgical instrument 77, determine the overlay related to the operation of the surgical instrument 77, and combine the aspect of the tissue in the surgical area with the functional data from the surgical instrument 77. The augmented images indicate alerts relative to device performance considerations, alerts of incompatible usage, alerts on incomplete capture. Incompatible usage includes tissue out range conditions and tissue incorrectly balanced within the jaws of the end effector. Additional augmented images provide an indication of collateral events including indication of tissue tension and indication of foreign object detection. Other augmented images indicate device status overlays and instrument indication.

FIG. 10 illustrates a system 83 for augmenting images of a surgical field with information using an AR display 89, according to one aspect of this disclosure. The system 83 may be used to perform the techniques described hereinbelow, for example, by using the processor 85. The system 83 includes one aspect of an AR device 66 that may be in communication with a database 93. The AR device 66 includes a processor 85, memory 87, an AR display 89, and a camera 79. The AR device 66 may include a sensor 90, a speaker 91, and/or a haptic controller 92. The database 93 may include image storage 94 or preoperative plan storage 95.

The processor 85 of the AR device 66 includes an augmented reality modeler 86. The augmented reality modeler 86 may be used by the processor 85 to create the augmented reality environment. For example, the augmented reality modeler 86 may receive images of the instrument in a surgical field, such as from the camera 79 or sensor 90, and create the augmented reality environment to fit within a display image of the surgical field of view. In another example, physical objects and/or date may be overlaid on the surgical field of view and/or the surgical instruments images and the augmented reality modeler 86 may use physical objects and data to present the augmented reality display of virtual object s and/or data in the augmented reality environment. For example, the augmented reality modeler 86 may use or detect an instrument at a surgical site of the patient and present a virtual object and/or data on the surgical instrument and/or an image of the surgical site in the surgical field of view captured by the camera 79. The AR display 89 may display the AR environment overlaid on a real environment. The display 89 may show a virtual object and/or data, using the AR device 66, such as in a fixed position in the AR environment.

The AR device 66 may include a sensor 90, such as an infrared sensor. The camera 79 or the sensor 90 may be used to detect movement, such as a gesture by a surgeon or other user, that may be interpreted by the processor 85 as attempted or intended interaction by the user with the virtual target. The processor 85 may identify an object in a real environment, such as through processing information received using the camera 79. In other aspects, the sensor 90 may be a tactile, audible, chemical, or thermal sensor to generate corresponding signals that may combined with various data feeds to create the augmented environment. The sensor 90 may include binaural audio sensors (spatial sound), inertial measurement (accelerometer, gyroscope, magnetometer) sensors, environmental sensors, depth camera sensors, hand and eye tracking sensors, and voice command recognition functions.

The AR display 89, for example during a surgical procedure, may present, such as within a surgical field while permitting the surgical field to be viewed through the AR display 89, a virtual feature corresponding to a physical feature hidden by an anatomical aspect of a patient. The virtual feature may have a virtual position or orientation corresponding to a first physical position or orientation of the physical feature. In an example, the virtual position or orientation of the virtual feature may include an offset from the first physical position or orientation of the physical feature. The offset may include a predetermined distance from the augmented reality display, a relative distance from the augmented reality display to the anatomical aspect, or the like.

In one example, the AR device 66 may be an individual AR device. In one aspect, the AR device 66 may be a HoloLens 2 AR device manufactured by Microsoft of Redmond, Wash. This AR device 66 includes a visor with lenses and binaural audio features (spatial sound), inertial measurement (accelerometer, gyroscope, magnetometer), environmental sensors, depth camera, and video camera, hand and eye tracking, and voice command recognition functions. It provides an improved field of view with high resolution by using mirrors to direct waveguides in front of wearer's eyes. Images can be enlarged by changing angles of mirrors. It also provides eye tracking to recognize users and adjust lens widths for specific users.

In another example, the AR device 66 may be a Snapchat Spectacles 3 AR device. This AR device provides the ability to capture paired images and recreate 3D depth mapping, add in virtual effects, and replay 3D videos. The AR device includes two HD cameras to capture 3D photos and videos at 60 fps—while four built-in microphones record immersive, high-fidelity audio. Images from both cameras combine to build out a geometric map of the real world around the user to provide a new sense of depth perception. Photos and videos may be wirelessly synchronized to external display devices.

In yet another example, the AR device 66 may be a Glass 2 AR device by Google. This AR device provides inertial measurement (accelerometer, gyroscope, magnetometer) information overlaid on lens (out of view) to supplement information.

In another example, the AR device 66 may be an Echo Frames AR device by Amazon. This AR device does not have cameras/displays. A microphone and speaker are linked to Alexa. This AR device provides less functionality than a heads-up display.

In yet another example, the AR device 66 may be a Focals AR device by North (Google). This AR device provides notification pusher/smartwatch analog; inertial measurement, screen overlay of information (weather, calendar, messages), and voice control (Alexa) integration. This AR device also provides basic heads-up display functionality.

In another example, the AR device 66 may be an Nreal AR device. This AR device includes spatial sound, two environmental cameras, a photo camera, IMU (accelerometer, gyroscope), ambient light sensor, proximity sensor functionality. A nebula projects application information on lenses.

In various other examples, the AR device 66 may be any one of the following commercially available AR devices: Magic Leap 1, Epson Moverio, Vuzix Blade AR, ZenFone AR, Microsoft AR glasses prototype, EyeTap to create collinear light to that of the environment directly into the retina. A beam splitter makes the same light seen by the eye available to the computer to process and overlay information, for example. AR visualization systems include HUD, contact lenses, glasses, virtual reality (VR) headsets, virtual retinal display, on in operating room displays, and/or smart contact lenses (bionic lenses).

Multi-user interfaces for the AR device 66 include virtual retinal displays such as raster displays drawn directly on retinas instead of on a screen in front of the eye, smart televisions, smart phones, and/or spatial displays such as Sony spatial display systems.

Other AR technology may include, for example, AR capture devices and software applications, AR creation devices and software applications, and AR cloud devices and software applications. AR capture devices and software applications include, for example, Apple Polycam app, Ubiquity 6 (Mirrorworld using Display.land app)—users can scan and get 3d image of real world (to create 3D model). AR creation devices and software applications include, for example, Adobe Aero, Vuforia, ARToolKit, Google ARCore, Apple ARKit, MAXST, Aurasma, Zappar, Blippar. AR cloud devices and software applications include, for example, Facebook, Google (world geometry, objection recognition, predictive data), Amazon AR Cloud (commerce), Microsoft Azure, Samsung Project Whare, Niantic, Magic Leap.

Situational awareness is the ability of some aspects of a surgical system to determine or infer information related to a surgical procedure from data received from databases and/or instruments. The information can include the type of procedure being undertaken, the type of tissue being operated on, or the body cavity that is the subject of the procedure. With the contextual information related to the surgical procedure, the surgical system can, for example, improve the manner in which it controls the modular devices (e.g., a robotic arm and/or robotic surgical tool) that are connected to it and provide contextualized information or suggestions to the surgeon during the course of the surgical procedure.

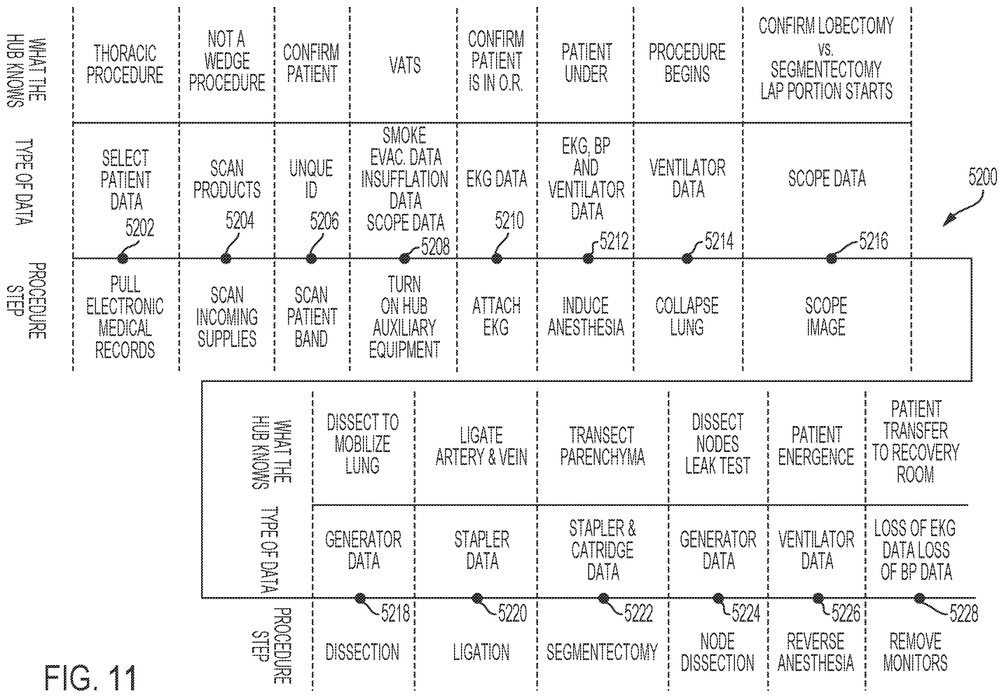

FIG. 11 illustrates a timeline of a situational awareness surgical procedure. FIG. 11 illustrates a timeline 5200 of an illustrative surgical procedure and the contextual information that a surgical hub 5104 can derive from the data received from the data sources 5126 at each step in the surgical procedure. The timeline 5200 depicts the typical steps that would be taken by the nurses, surgeons, and other medical personnel during the course of a lung segmentectomy procedure, beginning with setting up the operating theater and ending with transferring the patient to a post-operative recovery room. The situationally aware surgical hub 5104 receives data from the data sources 5126 throughout the course of the surgical procedure, including data generated each time medical personnel utilize a modular device 5102 that is paired with the surgical hub 5104. The surgical hub 5104 can receive this data from the paired modular devices 5102 and other data sources 5126 and continually derive inferences (i.e., contextual information) about the ongoing procedure as new data is received, such as which step of the procedure is being performed at any given time. The situational awareness system of the surgical hub 5104 is able to, for example, record data pertaining to the procedure for generating reports, verify the steps being taken by the medical personnel, provide data or prompts (e.g., via a display screen) that may be pertinent for the particular procedural step, adjust modular devices 5102 based on the context (e.g., activate monitors, adjust the FOV of the medical imaging device, or change the energy level of an ultrasonic surgical instrument or RF electrosurgical instrument), and take any other such action described above.

First 5202, the hospital staff members retrieve the patient's EMR from the hospital's EMR database. Based on select patient data in the EMR, the surgical hub 5104 determines that the procedure to be performed is a thoracic procedure.

Second 5204, the staff members scan the incoming medical supplies for the procedure. The surgical hub 5104 cross-references the scanned supplies with a list of supplies that are utilized in various types of procedures and confirms that the mix of supplies corresponds to a thoracic procedure. Further, the surgical hub 5104 is also able to determine that the procedure is not a wedge procedure (because the incoming supplies either lack certain supplies that are necessary for a thoracic wedge procedure or do not otherwise correspond to a thoracic wedge procedure).

Third 5206, the medical personnel scan the patient band via a scanner 5128 that is communicably connected to the surgical hub 5104. The surgical hub 5104 can then confirm the patient's identity based on the scanned data.

Fourth 5208, the medical staff turns on the auxiliary equipment. The auxiliary equipment being utilized can vary according to the type of surgical procedure and the techniques to be used by the surgeon, but in this illustrative case they include a smoke evacuator, insufflator, and medical imaging device. When activated, the auxiliary equipment that are modular devices 5102 can automatically pair with the surgical hub 5104 that is located within a particular vicinity of the modular devices 5102 as part of their initialization process. The surgical hub 5104 can then derive contextual information about the surgical procedure by detecting the types of modular devices 5102 that pair with it during this pre-operative or initialization phase. In this particular example, the surgical hub 5104 determines that the surgical procedure is a VATS procedure based on this particular combination of paired modular devices 5102. Based on the combination of the data from the patient's EMR, the list of medical supplies to be used in the procedure, and the type of modular devices 5102 that connect to the hub, the surgical hub 5104 can generally infer the specific procedure that the surgical team will be performing. Once the surgical hub 5104 knows what specific procedure is being performed, the surgical hub 5104 can then retrieve the steps of that procedure from a memory or from the cloud and then cross-reference the data it subsequently receives from the connected data sources 5126 (e.g., modular devices 5102 and patient monitoring devices 5124) to infer what step of the surgical procedure the surgical team is performing.

Fifth 5210, the staff members attach the EKG electrodes and other patient monitoring devices 5124 to the patient. The EKG electrodes and other patient monitoring devices 5124 are able to pair with the surgical hub 5104. As the surgical hub 5104 begins receiving data from the patient monitoring devices 5124, the surgical hub 5104 thus confirms that the patient is in the operating theater.

Sixth 5212, the medical personnel induce anesthesia in the patient. The surgical hub 5104 can infer that the patient is under anesthesia based on data from the modular devices 5102 and/or patient monitoring devices 5124, including EKG data, blood pressure data, ventilator data, or combinations. Upon completion of the sixth step 5212, the pre-operative portion of the lung segmentectomy procedure is completed and the operative portion begins.

Seventh 5214, the patient's lung that is being operated on is collapsed (while ventilation is switched to the contralateral lung). The surgical hub 5104 can infer from the ventilator data that the patient's lung has been collapsed. The surgical hub 5104 can infer that the operative portion of the procedure has commenced as it can compare the detection of the patient's lung collapsing to the expected steps of the procedure (which can be accessed or retrieved previously) and thereby determine that collapsing the lung is the first operative step in this particular procedure.

Eighth 5216, the medical imaging device 5108 (e.g., a scope) is inserted and video from the medical imaging device is initiated. The surgical hub 5104 receives the medical imaging device data (i.e., still image data or live streamed video in real time) through its connection to the medical imaging device. Upon receipt of the medical imaging device data, the surgical hub 5104 can determine that the laparoscopic portion of the surgical procedure has commenced. Further, the surgical hub 5104 can determine that the particular procedure being performed is a segmentectomy, as opposed to a lobectomy (note that a wedge procedure has already been discounted by the surgical hub 5104 based on data received at the second step 5204 of the procedure). The data from the medical imaging device 124 (FIG. 2) can be utilized to determine contextual information regarding the type of procedure being performed in a number of different ways, including by determining the angle at which the medical imaging device is oriented with respect to the visualization of the patient's anatomy, monitoring the number or medical imaging devices being utilized (i.e., that are activated and paired with the surgical hub 5104), and monitoring the types of visualization devices utilized.

For example, one technique for performing a VATS lobectomy places the camera in the lower anterior corner of the patient's chest cavity above the diaphragm, whereas one technique for performing a VATS segmentectomy places the camera in an anterior intercostal position relative to the segmental fissure. Using pattern recognition or machine learning techniques, for example, the situational awareness system can be trained to recognize the positioning of the medical imaging device according to the visualization of the patient's anatomy. As another example, one technique for performing a VATS lobectomy utilizes a single medical imaging device, whereas another technique for performing a VATS segmentectomy utilizes multiple cameras. As yet another example, one technique for performing a VATS segmentectomy utilizes an infrared light source (which can be communicably coupled to the surgical hub as part of the visualization system) to visualize the segmental fissure, which is not utilized in a VATS lobectomy. By tracking any or all of this data from the medical imaging device 5108, the surgical hub 5104 can thereby determine the specific type of surgical procedure being performed and/or the technique being used for a particular type of surgical procedure.

Ninth 5218, the surgical team begins the dissection step of the procedure. The surgical hub 5104 can infer that the surgeon is in the process of dissecting to mobilize the patient's lung because it receives data from the RF or ultrasonic generator indicating that an energy instrument is being fired. The surgical hub 5104 can cross-reference the received data with the retrieved steps of the surgical procedure to determine that an energy instrument being fired at this point in the process (i.e., after the completion of the previously discussed steps of the procedure) corresponds to the dissection step.

Tenth 5220, the surgical team proceeds to the ligation step of the procedure. The surgical hub 5104 can infer that the surgeon is ligating arteries and veins because it receives data from the surgical stapling and cutting instrument indicating that the instrument is being fired. Similarly to the prior step, the surgical hub 5104 can derive this inference by cross-referencing the receipt of data from the surgical stapling and cutting instrument with the retrieved steps in the process.

Eleventh 5222, the segmentectomy portion of the procedure is performed. The surgical hub 5104 infers that the surgeon is transecting the parenchyma based on data from the surgical instrument, including data from a staple cartridge. The cartridge data may correspond to size or type of staple being fired by the instrument. The cartridge data can indicate the type of tissue being stapled and/or transected for different types of staples utilized in different types of tissues. The type of staple being fired is utilized for parenchyma or other tissue types to allow the surgical hub 5104 to infer that the segmentectomy procedure is being performed.

Twelfth 5224, the node dissection step is then performed. The surgical hub 5104 can infer that the surgical team is dissecting the node and performing a leak test based on data received from the generator indicating that an RF or ultrasonic instrument is being fired. For this particular procedure, an RF or ultrasonic instrument being utilized after parenchyma was transected corresponds to the node dissection step, which allows the surgical hub 5104 to make this inference. It should be noted that surgeons regularly switch back and forth between surgical stapling/cutting instruments and surgical energy (i.e., RF or ultrasonic) instruments depending upon the particular step in the procedure because different instruments are better adapted for particular tasks. Therefore, the particular sequence in which the stapling/cutting instruments and surgical energy instruments are used can indicate what step of the procedure the surgeon is performing. Upon completion of the twelfth step 5224, the incisions and closed up and the post-operative portion of the procedure begins.

Thirteenth 5226, the patient's anesthesia is reversed. The surgical hub 5104 can infer that the patient is emerging from the anesthesia based on the ventilator data (i.e., the patient's breathing rate begins increasing), for example.

Lastly, fourteenth 5228, the medical personnel remove the various patient monitoring devices 5124 from the patient. The surgical hub 5104 can thus infer that the patient is being transferred to a recovery room when the hub loses EKG, BP, and other data from the patient monitoring devices 5124. The surgical hub 5104 can determine or infer when each step of a given surgical procedure is taking place according to data received from the various data sources 5126 that are communicably coupled to the surgical hub 5104.

In addition to utilizing the patient data from EMR database(s) to infer the type of surgical procedure that is to be performed, as illustrated in the first step 5202 of the timeline 5200 depicted in FIG. 11, the patient data can also be utilized by a situationally aware surgical hub 5104 to generate control adjustments for the paired modular devices 5102.

Surgical displays (e.g., displays 7, 9, 19, 35, 62, 65, 66, 67, and 89) play an important function within the operating room, by providing useful information to a clinician (e.g., surgeon, surgical staff, etc.) that can be used to, among other things, assess the progress of a surgical procedure, determine subsequent steps to take in the surgical procedure, monitor patent vital signs, etc. The displays need to be large enough that the information being provided can be seen, yet not so large as to be overbearing and obstruct workflow or movement in a crowded operating room.

For example, an imaging device, such as one of the many imaging devices described elsewhere herein, is used to capture a livestream of a surgical field during a surgical procedure. A display shows this livestream captured by the imaging device such that the clinician can view the surgical field during the surgical procedure.

During the course of the surgical procedure, information that is relevant to or associated with the surgical procedure can be overlaid onto the livestream on the display. For example, an electrocardiogram (EKG) monitors a patient's heart rate during the surgical procedure and the monitored heart rate is overlaid on the livestream such that the clinician can ensure that the patient is stable.

Various other sensors, detectors, modules, etc. monitor other parameters over the course of the surgical procedure and information associated with these parameters can also be overlaid onto the display. However, some overlaid information may be of more significance than other overlaid information. As an example, when a clinician is manipulating tissue with an end effector of a surgical instrument, information regarding how much force is being applied to the tissue with the end effector is relevant to monitor so as to ensure the tissue isn't being unintentionally damaged.

However, owing to the amount of information being overlaid on the display, more important information, such as a force being applied to the tissue, may be overlooked or missed by the clinician. This abundance of competing information can cause the surgeon to become overwhelmed with information that may be detrimental to their ability to adequately perform the surgical procedure, which can prove costly to the patient. Accordingly, there is a need to prioritize, control and/or limit the amount of data that is being overlaid on the display.

### Automatic Prompting for Pairing Wireless Devices to Simplify Workflow

Wireless communication between surgical devices and other equipment in the operating room (OR) suite can enable additional insights for users. In order to establish wireless communication, pairing these devices is necessary. For example, a surgical device (described generally in FIGS. 1-11) using Bluetooth Low Energy (BLE) needs to be paired to a general purpose computer (GPC), e.g. surgical hub 6 or surgical hub 56 (FIGS. 1-11), to enable on screen overlays of relevant information. Therefore, a pairing process that is simple, intuitive and/or easily understandable is desired to reduce confusion and simplify workflow for pairing surgical devices to other equipment in the OR. The following description provides an example of a surgical device using BLE pairing to a general purpose computer will be used. However, this concept applies to other surgical devices and OR equipment, as well as other wireless technologies beyond BLE.

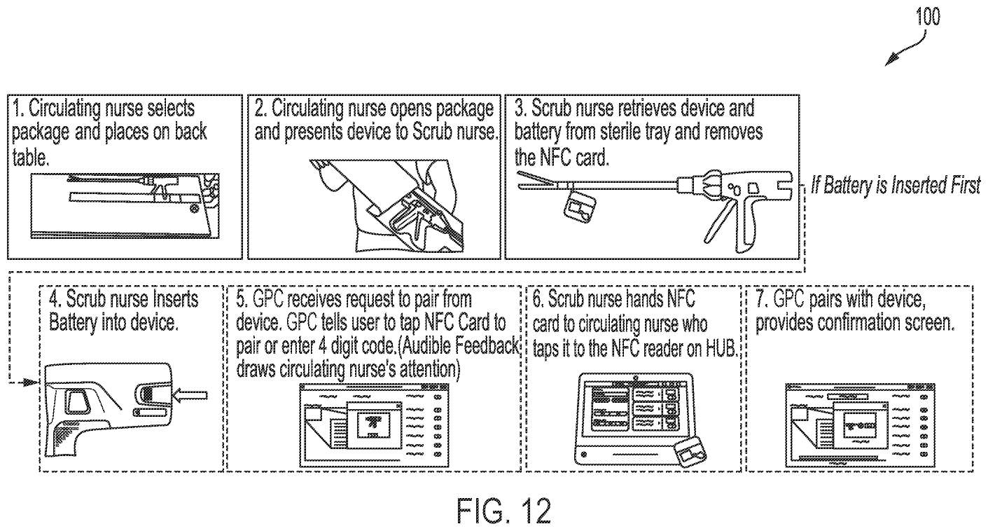

FIG. 12 illustrates a pairing workflow 100 if a battery is first inserted into the surgical device/instrument before pairing is established according to at least one aspect of the present disclosure. In this technique, when the battery of the surgical device/instrument is plugged in, the surgical device/instrument sends out a request to pair to the general purpose computer, e.g. surgical hub 6 or surgical hub 56. This request may be transmitted over BLE such as through its advertising mechanism on startup, or could similarly be transmitted by another device mechanism (alternative wireless technology, local near field communication [NFC] chip on the device itself, etc.). The general purpose computer receives this request and then displays on a screen instructions to the user. For example, the on screen instructions may include tapping an RFID card provided with the surgical device/instrument to an RFID reader of the general purpose computer or entering a multiple digit code that is printed on the surgical device/instrument into a user interface of the general purpose computer. In at least one aspect, the on screen instructions may include both tapping an RFID card provided with the surgical device/instrument to an RFID reader of the general purpose computer and entering a multiple digit code that is printed on the surgical device/instrument into a user interface of the general purpose computer.

As shown in FIG. 12, if the battery is inserted first in the surgical device/instrument, the circulating nurse selects a package from storage and places it on the back table. The circulating nurse opens the package and presents the surgical device/instrument to the scrub nurse. The scrub nurse retrieves the surgical device/instrument and battery from the sterile tray and removes the NFC card from the surgical device/instrument. When the scrub nurse inserts the battery into the surgical device/instrument, the general purpose computer, e.g. surgical hub 6 or surgical hub 56, receives a request to pair from the surgical device/instrument. In at least one aspect, the surgical device/instrument transmits the request upon the surgical device/instrument powering on due to the insertion of the battery. The general purpose computer tells the user to tap the NFC card to pair and/or enter a 3, 4, or any multi-digit code. In at least one aspect, the general purpose computer plays an audible feedback which can draw the circulating nurse's attention to displayed instructions. For example the audible feedback can be played in response to the instructions being transmitted. The scrub nurse hands the NFC card to the circulating nurse who taps it to the NFC reader on the general purpose computer. In at least one aspect, the general purpose computer receives identification data of the surgical device from the NFC card through the NFC card reader. The general purpose computer pairs with the surgical device/instrument and provides a confirmation screen.

Additionally, the general purpose computer may use feedback mechanisms to draw the user's attention to the display to pair the specific device (such as but not limited to audio tones, visual ques/flashing, or haptic buzzing).

The pairing instructions shown on screen can be specific to the type of instrument attempting to pair with the general purpose computer. For example, if a stapler is attempting to pair, an image of the stapler will appear on the screen—or—if another surgical device/instrument is attempting to pair, an image of the other surgical device/instrument will appear on the screen.

Other methods of pairing require the user to remember to pair the device (using an RFID tap or code input). This method detects when pairing is possible and prompts the user to pair, which reduces confusion and increases the likelihood that pairing will happen.

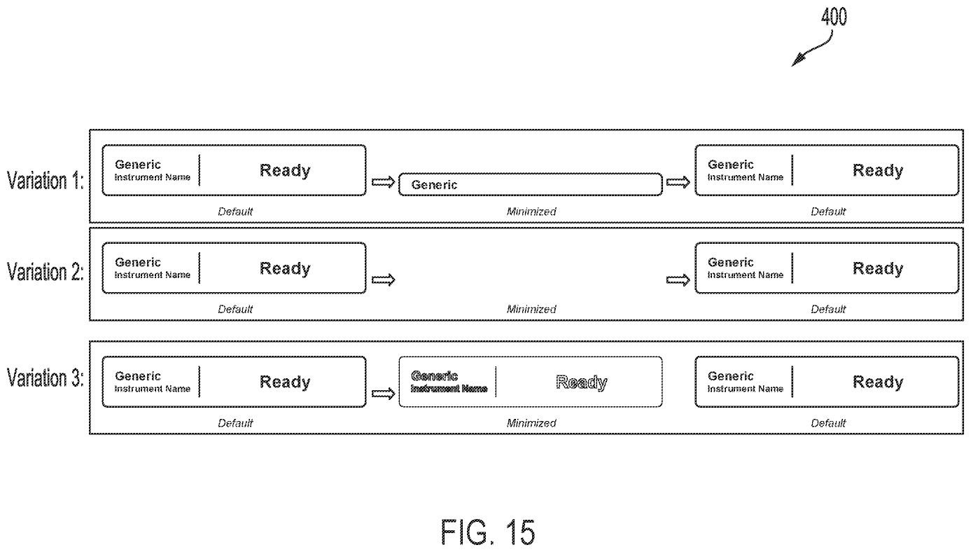

In at least one aspect, the scrub nurse or the circulating nurse enters into the general purpose computer identifying information, e.g. surgeon's name, identification number, etc., for the surgeon performing the surgical procedure. In an alternative aspect, the general purpose computer accesses a hospital database to determine the surgeon performing the surgical procedure. The general purpose computer retrieves preferences of the surgeon from a database of surgeon preferences using identification data of the surgeon, e.g. employee id, name, etc. In at least one aspect, the preferences of the surgeon include control parameters of the surgical device/instrument paired with the general purpose computer, where the control parameters are based on the surgical procedure being performed. For example, the surgeon preferences for the control parameters for the surgical device/instrument could be different based on the type of surgical procedure being performed. In at least one aspect, the general purpose computer transmits the control parameters to the surgical device/instrument. In at least one aspect, the control parameters include control settings for the surgical device, e.g. custom control preferences for buttons, motor current thresholds, motor velocity thresholds, overlay configurations relating to the surgical device, and any other parameter that can be set or changed by the surgeon. In an additional aspect, the overlay configurations are for an overlay, as described in regard to FIGS. 15-30, of data from the surgical device/instrument paired with the general purpose computer.

In this aspect, the general purpose computer displays the overlay based on the overlay configuration on a primary surgical display, where the overlay is displayed overtop of information on the primary surgical display. For example, the overlay can be displayed overtop of camera data of the surgical site. In at least one aspect, the overlay includes real-time data from the surgical device/instrument. For example, the overlay could include any data sensed by the surgical device/instrument, e.g. force to close an end effector on tissue, force to cut tissue, energy required to cut tissue, etc. In at least one aspect, the overlay also includes information or data calculated by the general purpose computer or surgical device/instrument, e.g. a warning about a parameter exceeding a threshold can be provided in the overlay. For example, the general purpose computer can monitor data from the surgical device/instrument, e.g. force on a motor, motor current, energy used by an electrosurgical device/instrument, etc. The general purpose computer can compare the data from the surgical device to a predetermined threshold and provide a warning to the surgeon through the overlay upon the data exceeding a predetermined threshold.

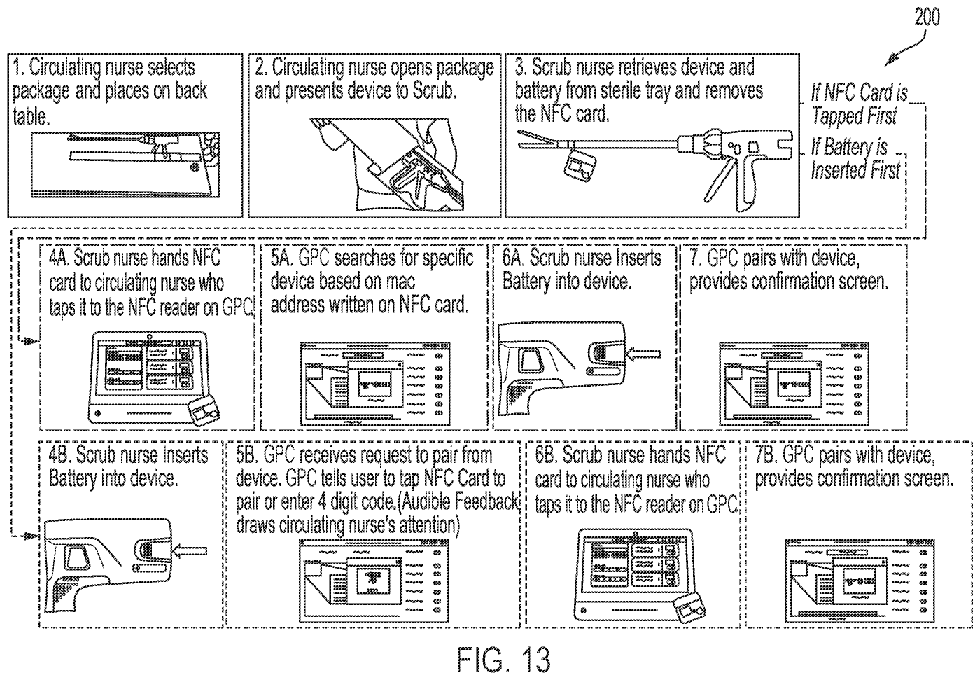

FIG. 13 illustrates a pairing workflow 200 if a NFC card is first tapped on the general purpose computer, e.g. surgical hub 6 or surgical hub 56, before pairing is established according to at least one aspect of the present disclosure. Accordingly, if a NFC card is first tapped on the general purpose computer, the scrub nurse hands the NFC card to the circulating nurse who taps it to the NFC reader on the general purpose computer. The general purpose computer searches for a specific surgical device/instrument based on the identification data, e.g. media access control (MAC) address, written on the NFC card. In at least one aspect, the general purpose computer searches through a database of identification data, e.g. MAC addresses, of a plurality of surgical devices to determine the specific surgical device or the type of surgical device. The scrub nurse then inserts the battery into the surgical device/instrument. In at least one aspect, the general purpose computer determines that the surgical device/instrument has turned on. For example, the general purpose computer could receive a request to pair with the surgical device/instrument, which allows the general purpose computer to determine that the surgical device/instrument has turned on. The general purpose computer then pairs with the surgical device/instrument and provides a confirmation screen.

In at least one aspect, the scrub nurse or the circulating nurse enters into the general purpose computer identifying information, e.g. surgeon's name, identification number, etc., for the surgeon performing the surgical procedure. In an alternative aspect, the general purpose computer accesses a hospital database to determine the surgeon performing the surgical procedure. The general purpose computer retrieves preferences of the surgeon from a database of surgeon preferences using identification data of the surgeon, e.g. employee id, name, etc. In at least one aspect, the preferences of the surgeon include control parameters of the surgical device/instrument paired with the general purpose computer, where the control parameters are based on the surgical procedure being performed. For example, the surgeon preferences for the control parameters for the surgical device/instrument could be different based on the type of surgical procedure being performed. In at least one aspect, the general purpose computer transmits the control parameters to the surgical device/instrument. In at least one aspect, the control parameters include control settings for the surgical device, e.g. custom control preferences for buttons, motor current thresholds, motor velocity thresholds, overlay configurations relating to the surgical device, and any other parameter that can be set or changed by the surgeon. In an additional aspect, the overlay configurations are for an overlay, as described in regard to FIGS. 15-30, of data from the surgical device/instrument paired with the general purpose computer.

In this aspect, the general purpose computer displays the overlay based on the overlay configuration on a primary surgical display, where the overlay is displayed overtop of information on the primary surgical display. For example, the overlay can be displayed overtop of camera data of the surgical site. In at least one aspect, the overlay includes real-time data from the surgical device/instrument. For example, the overlay could include any data sensed by the surgical device/instrument, e.g. force to close an end effector on tissue, force to cut tissue, energy required to cut tissue, etc. In at least one aspect, the overlay also includes information or data calculated by the general purpose computer or surgical device/instrument, e.g. a warning about a parameter exceeding a threshold can be provided in the overlay. For example, the general purpose computer can monitor data from the surgical device/instrument, e.g. force on a motor, motor current, energy used by an electrosurgical device/instrument, etc. The general purpose computer can compare the data from the surgical device to a predetermined threshold and provide a warning to the surgeon through the overlay upon the data exceeding a predetermined threshold.

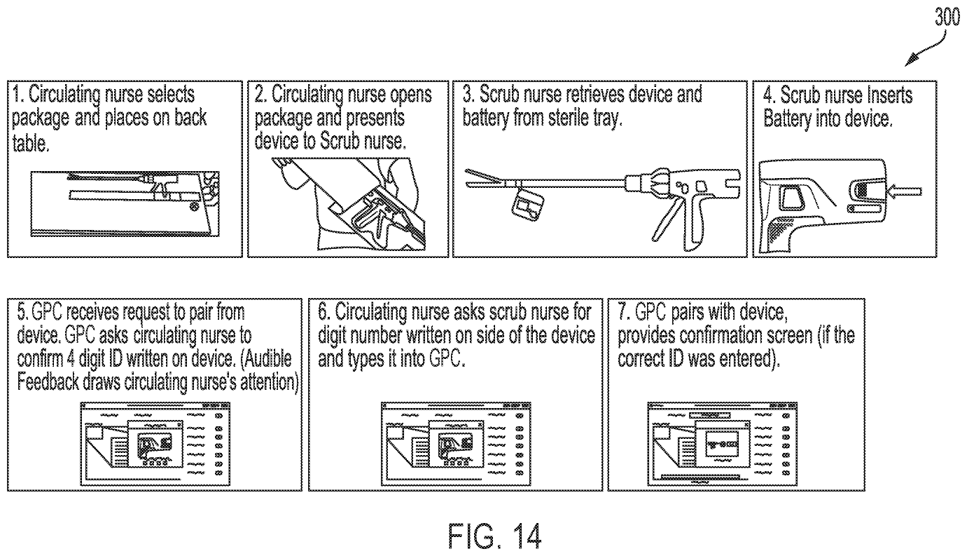

FIG. 14 illustrates a multi digit confirmation pairing workflow 300 according to at least one aspect of the present disclosure. The circulating nurse selects a package from storage and places it on the back table. The circulating nurse opens the package and presents the surgical device/instrument to the scrub nurse. The scrub nurse retrieves the surgical device/instrument and battery from the sterile tray. The scrub nurse inserts the battery into the surgical device/instrument. The general purpose computer, e.g. surgical hub 6 or surgical hub 56, receives a request to pair from the surgical device/instrument. The general purpose computer asks the circulating nurse to confirm a multi digit identification number (e.g., 3, 4, 5, 6, or any digit identification number) written on the surgical device/instrument (audible feedback can draw the circulating nurse's attention). The circulating nurse asks the scrub nurse for the multi digit number written on the side of the surgical device/instrument and types it into a user interface of the general purpose computer. If the correct multi digit number was entered, then the general purpose computer and the surgical device/instrument pair.

In at least one aspect, the scrub nurse or the circulating nurse enters into the general purpose computer identifying information, e.g. surgeon's name, identification number, etc., for the surgeon performing the surgical procedure. In an alternative aspect, the general purpose computer accesses a hospital database to determine the surgeon performing the surgical procedure. The general purpose computer retrieves preferences of the surgeon from a database of surgeon preferences using identification data of the surgeon, e.g. employee id, name, etc. In at least one aspect, the preferences of the surgeon include control parameters of the surgical device/instrument paired with the general purpose computer, where the control parameters are based on the surgical procedure being performed. For example, the surgeon preferences for the control parameters for the surgical device/instrument could be different based on the type of surgical procedure being performed. In at least one aspect, the general purpose computer transmits the control parameters to the surgical device/instrument. In at least one aspect, the control parameters include control settings for the surgical device, e.g. custom control preferences for buttons, motor current thresholds, motor velocity thresholds, overlay configurations relating to the surgical device, and any other parameter that can be set or changed by the surgeon. In an additional aspect, the overlay configurations are for an overlay, as described in regard to FIGS. 15-30, of data from the surgical device/instrument paired with the general purpose computer.

In this aspect, the general purpose computer displays the overlay based on the overlay configuration on a primary surgical display, where the overlay is displayed overtop of information on the primary surgical display. For example, the overlay can be displayed overtop of camera data of the surgical site. In at least one aspect, the overlay includes real-time data from the surgical device/instrument. For example, the overlay could include any data sensed by the surgical device/instrument, e.g. force to close an end effector on tissue, force to cut tissue, energy required to cut tissue, etc. In at least one aspect, the overlay also includes information or data calculated by the general purpose computer or surgical device/instrument, e.g. a warning about a parameter exceeding a threshold can be provided in the overlay. For example, the general purpose computer can monitor data from the surgical device/instrument, e.g. force on a motor, motor current, energy used by an electrosurgical device/instrument, etc. The general purpose computer can compare the data from the surgical device to a predetermined threshold and provide a warning to the surgeon through the overlay upon the data exceeding a predetermined threshold.

### Overlaid Information Dynamic Minimization

Adding overlays on the primary surgical display, e.g. displays 19, 35, 62, 65, 66, 67, and 89, can provide helpful information to the surgeon or OR staff. For example, users may only want to see information when it is contextually relevant. Sometimes, an instrument or device is connected to the system, e.g. surgical hub 6 or surgical hub 56, but is not actively being used, thereby taking up valuable laparoscopic/endoscopic video monitor space when it isn't needed. Enabling easy adjustment of the overlays helps surgeons maximize visualization of targeted anatomy while having access to relevant surgical device information on their primary surgical display when, where, and how they prefer it.

FIG. 15 illustrates instrument panel minimization 400 according to at least one aspect of the present disclosure. When connected devices are inactive or leave the surgical site, associated overlays are minimized to reduce the visual prominence of the overlays when not in use. Some variations of minimizing the overlay are described below.

Variation 1: After X seconds without detected interaction, the instrument panel minimizes to a smaller panel size that only shows a subset of information or reduces scale of information (e.g., such as instrument type or abbreviated instrument name, smaller font size, etc.).

Variation 2: After X seconds without detected interaction, the instrument panel disappears completely.

Variation 3: After X seconds without detected interaction, the instrument panel changes opacity (e.g. decreases opacity) so it's visually less prominent on screen.

In at least one aspect, minimizes the overlay can be a combination of the variations described above. For example, the overlay could be made smaller and have the opacity changed. The instrument panel returns to default view when an alert, activation, or other detected interaction occurs.

Interaction could be detected through sensors such as a temperature, position, proximity, infrared (IR), light, tilt, ultrasonic, or projected capacitive (PCAP) touch sensors or through user inputs such as a pressing of a switch, closing a trigger, interacting with a control touchpoint, etc. In at least one aspect, the data from sensors is received by a general purpose computer, e.g. surgical hub 6 or surgical hub 56.

Instrument interaction could be determined by detected interaction with the instrument via sensors or controls in the instrument or in the environment (e.g., instrument interaction could be detected through a secondary monitoring device such as a camera.) In at least one aspect, a general purpose computer determines instrument interaction. For example, a general purpose computer, e.g. surgical hub 6 or surgical hub 56, can analyze video data from a camera, sensor data, instrument input controls, and/or etc. to determine instrument interaction. In at least one aspect, the general purpose computer provides the overlays onto the display screen, e.g. displays 19, 35, 62, 65, 66, 67, and/or 89. For example, the general purpose computer can receive a live video feed from a camera, place an overlay on the live video feed, and then transmit the video feed and overlay to a display screen.

In at least one aspect, the general purpose computer receives real-time video data of a surgical site. The general purpose computer displays the real-time video data on a display. The general purpose computer determines that the surgical instrument is in the field of view of the camera and displays an overlay regarding the surgical instrument overtop of the real-time video data on the display. The general purpose computer monitors the real-time video data over time and determines that the surgical instrument is no longer in the field of view of the camera. The general purpose computer then automatically, minimizes, as discussed above, the overlay based on the surgical instrument no longer being within the field of view of the camera. Upon the surgical instrument coming back into the field of view of the camera, the general purpose computer enlarges the overlay, i.e. makes the overlay no longer be minimized.

In at least one aspect, the general purpose computer receives real-time video data of a surgical site. The general purpose computer displays the real-time video data on a display. The general purpose computer determines that surgical instrument is active by determining a detected interaction of the surgical instrument as discussed above. For example, a detected interaction could be received from sensor data on the instrument, sensor data detecting the instrument, camera data viewing the instrument, control data for the instrument, etc. Once the general purpose computer determines that surgical instrument is active, the general purpose computer displays an overlay regarding the surgical instrument overtop of the real-time video data on the display. The general purpose computer monitors the activity of the surgical instrument, e.g. by monitoring interactions with the surgical instrument. If the general purpose computer determines that the surgical instrument is no longer active, then the general purpose computer automatically, minimizes, as discussed above, the overlay based on the surgical instrument no longer being active. The general purpose computer can determine if the surgical instrument is active in a plurality of ways. One example, is for the general purpose computer to compare the time period from the last detected interaction of the surgical instrument and a predetermined threshold time. If a detected interaction of the surgical instrument has not occurred within a predetermined threshold time, then the surgical instrument can be determined as inactive. The predetermined threshold time can be based on the type of surgical instrument, the surgical procedure being performed, and/or ect. Upon the general purpose computer determining that the surgical instrument is again active, then the general purpose computer enlarges the overlay, i.e. makes the overlay no longer be minimized.

### Customize Overlays on Primary Surgical Display

Adding overlays on the primary surgical display, e.g. display 19, 35, 62, 65, 66, 67, and/or 89, can provide helpful information to the surgeon or OR staff. For example, some users may only want to see a subset of the available overlays, depending on clinician preference. As such, users desire a way to customize what information is displayed on the Primary Surgical Display. Adding information to the primary surgical display is a balancing act: too much information is distracting, too little information provides no additional value. Some unmet needs may include enabling easy adjustment of the overlays to help surgeons maximize visualization of targeted anatomy while having access to relevant surgical device information on their primary surgical display when, where, and how they prefer it.

A secondary user interface (called “Staff View” in the illustrations) can control which overlays are shown on the primary surgical display (laparoscopic/endoscopic monitor). In at least one aspect, the primary surgical display is part of the primary user interface for a surgeon and the secondary user interface is for a staff user, e.g. a nurse, technician, ect. The Staff View can also control how the overlays are displayed by adjusting position, scale, and amount of information shown for each overlay element. Individual pieces of overlaid information can be adjusted, or groups of information can be adjusted simultaneously through the Staff View Interface.

In at least one aspect, the Staff View can be load data from a selected overlay configuration, where a staff user can select an overlay configuration from a database that includes a plurality of stored overlay configurations. The selected overlay configured can be based on the type of surgical procedure being performed and the preferences of the surgeon performing the surgical procedure. The stored overlay configurations can be related to a surgeon's preferences for a type of surgical procedure. In an alternative aspect, the general purpose computer can automatically load data of an overlay configuration into the Staff View, where the overlay configuration is based on the surgeon performing the surgical procedure and the type of surgical procedure. For example, the general purpose computer can retrieve an overlay configuration from a database based on the surgeon performing the surgical procedure and the type of surgical procedure. The database can include overlay configurations that are based on a surgeon's preferences for a type of surgical procedure.

As changes are made on the Staff View, the changes are visible on the surgeon's primary surgical display, e.g. display 19, 35, 62, 65, 66, 67, and/or 89. The changes to the Staff View may be displayed instantaneously or displayed after a delay on the primary surgical display. In at least one aspect, the general purpose computer, e.g. surgical hub 6 or surgical hub 56, receives the overlay configuration from the Staff View Interface and then provides that overlay configuration onto the display screens. In at least one aspect, the changes to the overlay configuration can be used to create a new overlay configuration stored in the database or update an existing overlay configuration stored in the database.

In at least one aspect, the Staff View Interface is located on a display 7, 9 that allows a non-sterile operator to adjust the overlaid information for the surgeon. For example, the Staff View can be located outside of the sterile field in the operating room and a non-sterile operator could adjust the overlaid information for the surgeon from outside of the sterile field during a surgical procedure being performed by the surgeon in the sterile field. In an alternative aspect, the Staff View Interface is located on the display 35 of the general purpose computer and a sterile operator adjusts the overlaid information for the surgeon. In yet another alternative aspect, the Staff View Interface is located on the displayed on the main laparoscopic/endoscopic surgical display and a sterile operator, e.g. nurse, surgeon, etc., adjusts the overlaid information.

### Staff View Customization Screens

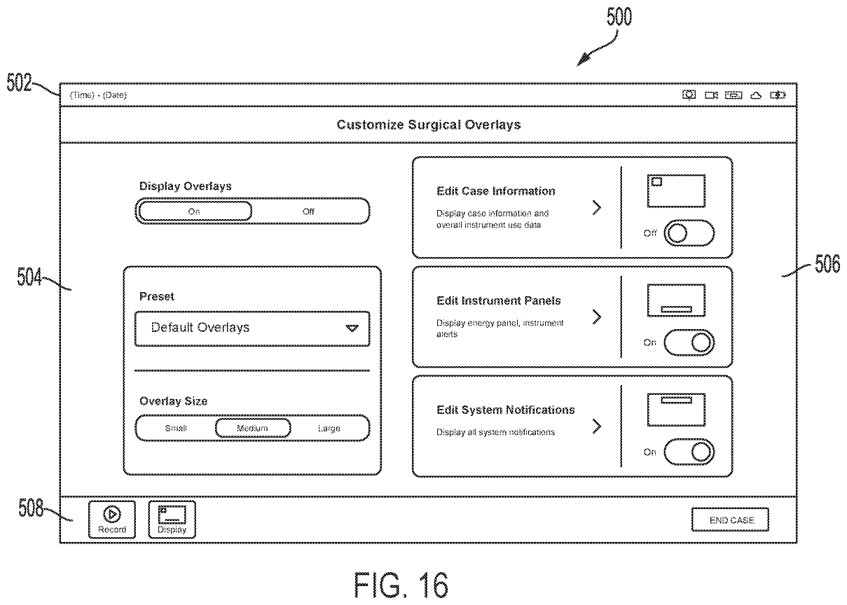

FIG. 16 illustrates a Staff View user interface control screen 500 that is displayed on the main laparoscopic/endoscopic surgical display, e.g. display 7, 9, 19, 35, 62, 65, 66, 67, and/or 89, according to at least one aspect of the present disclosure. A general purpose computer header 502 shows system wide information and connection status updates. A general display control 504 affects all overlays on a surgical display. For example, show/hide all overlays, set user presets, adjust overlay size on display, etc. A customize overlay panel 506 fine tunes content in each overlay section. For example, toggle visibility for each panel, an additional panel for more customization modal, and etc. A general purpose computer footer 508 allows a user to navigate between applications. In at least one aspect, the general purpose computer, e.g. surgical hub 6 or surgical hub 56, receives an input from the Staff View user interface and adjust the Staff View user interface based on the input. In at least one aspect, an overlay configuration can be loaded from and/or stored in a database that includes a plurality of overlays that are specific to a surgeon performing a surgical procedure. The general purpose computer can auto populate the Staff View based on an overlay configuration.

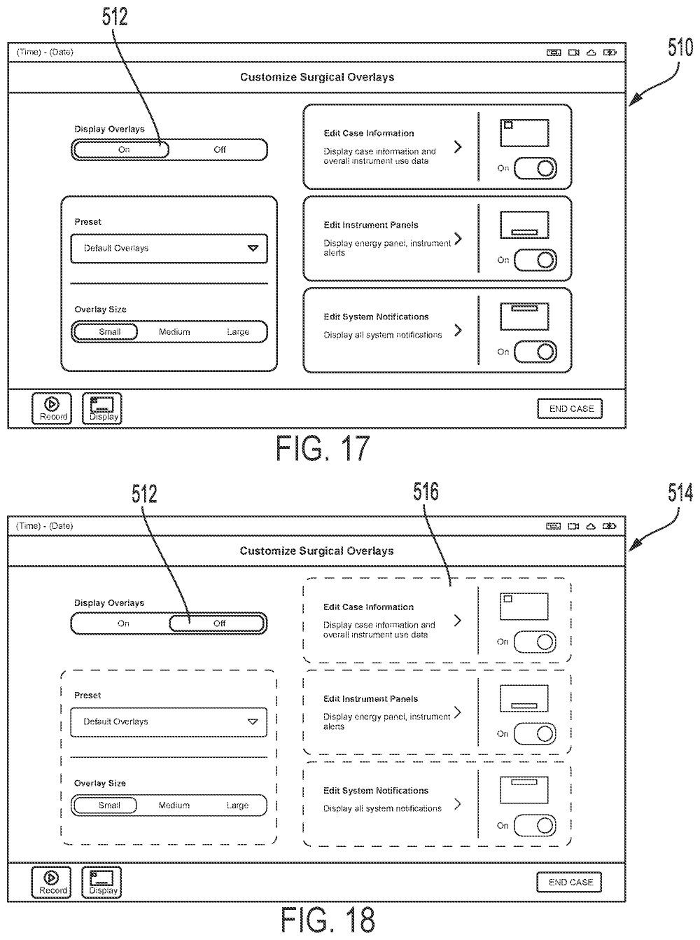

FIGS. 17-22 show behavior of general display controls. In at least one aspect, the display controls are received by a general purpose computer, e.g. surgical hub 6 or surgical hub 56, and are used to determine an overlay configuration for one or more displays, e.g. displays 7, 9, 19, 35, 62, 65, 66, 67, and/or 89. An overlay configuration can be specific to a surgeon's preferences for performing a surgical procedure and the overlay configuration can be stored in a database of overlay configurations based on surgeon preferences. For example, the general purpose computer can receive identification data of a surgeon performing a surgical procedure and can then retrieve an overlay configuration from a database based on the surgical procedure and the identification information of the surgeon. The general purpose computer can then display an overlay based on the overlay configuration on a display screen. This can allow the general purpose computer to automatically load an overlay configuration and display an overlay based on the surgeon's preferences for a surgical procedure. For example, the surgeon could have a preference on the data they desire to see from a surgical stapling device during a type of surgical procedure. FIGS. 17-22 illustrate general display controls that can be used to create an overlay configuration that can be stored in the database of overlay configurations.

FIG. 17 illustrates a display overlay toggle screen 510 with a display overlay virtual switch 512 in the “On” position according to at least one aspect of the present disclosure. When switched to “Off”, as shown on the display screen 514 in FIG. 18, the customize panels 516 become disabled.

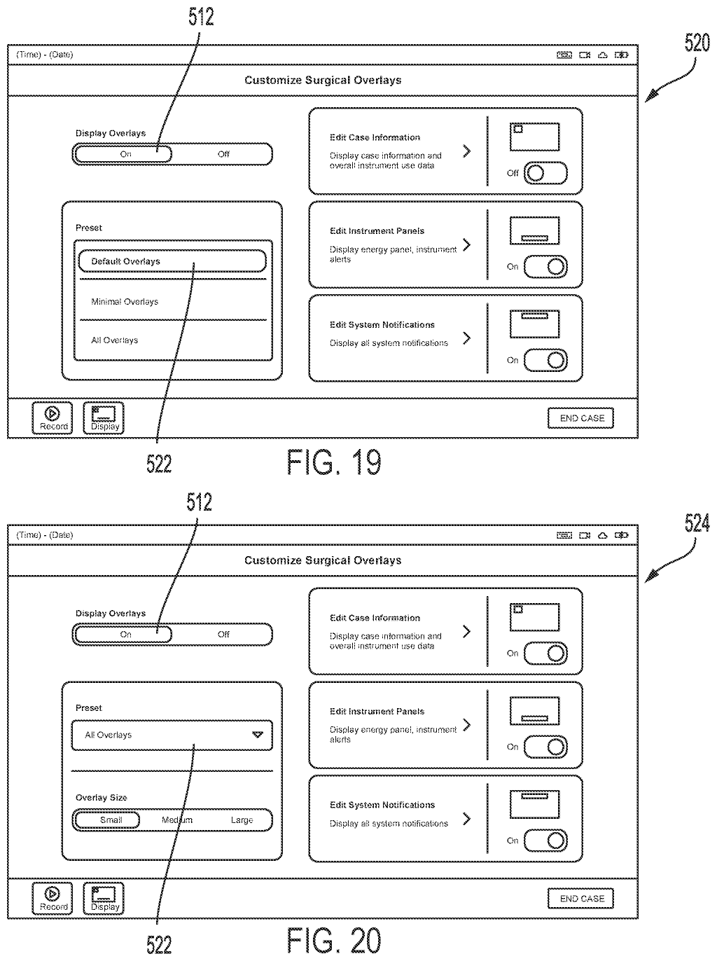

FIG. 19 illustrates a presets dropdown screen 520 according to at least one aspect of the present disclosure. Users can select presets 522 to quickly change multiple settings. Selecting a new preset 522 will override any previous changes. A new preset dropdown screen 524 is displayed after selecting the preset 522 as shown in FIG. 20.

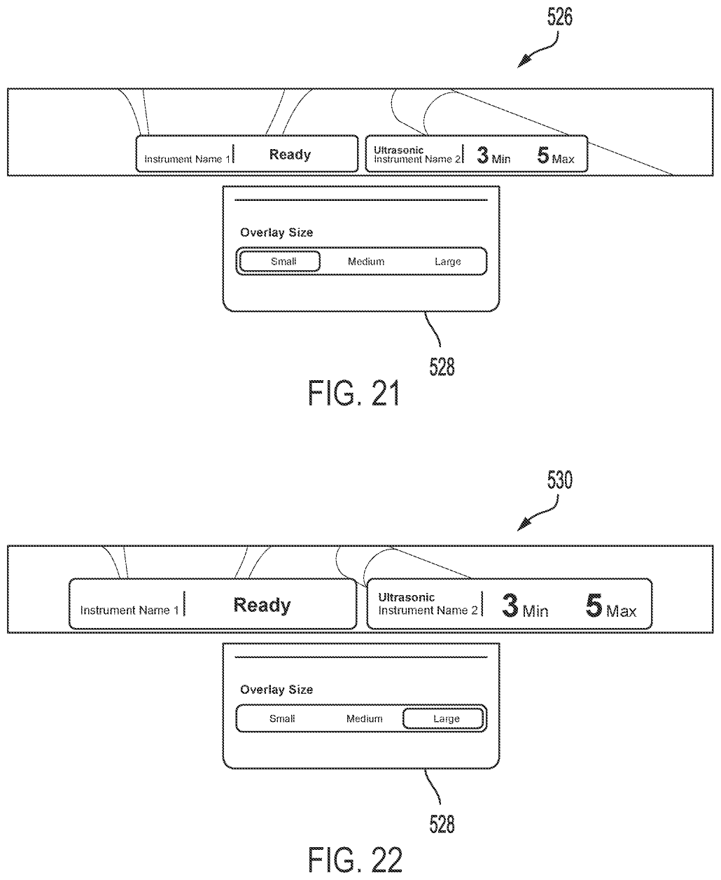

FIG. 21 illustrates a screen 526 with an overlay size selection screen 528 with the overlay size set to small according to at least one aspect of the present disclosure. Users can adjust the size of the panels on the Photoshop document (PSD). Medium is the default setting. Small is 0.8×, and large is 1.2× the default size. The overlay size of the screen 526 in FIG. 22 is set to large and the overlay size of the screen 530 in FIG. 21 is set to large. The adjustment may encompass any suitable size and by way of example may be selected as Small, Medium, and Large.

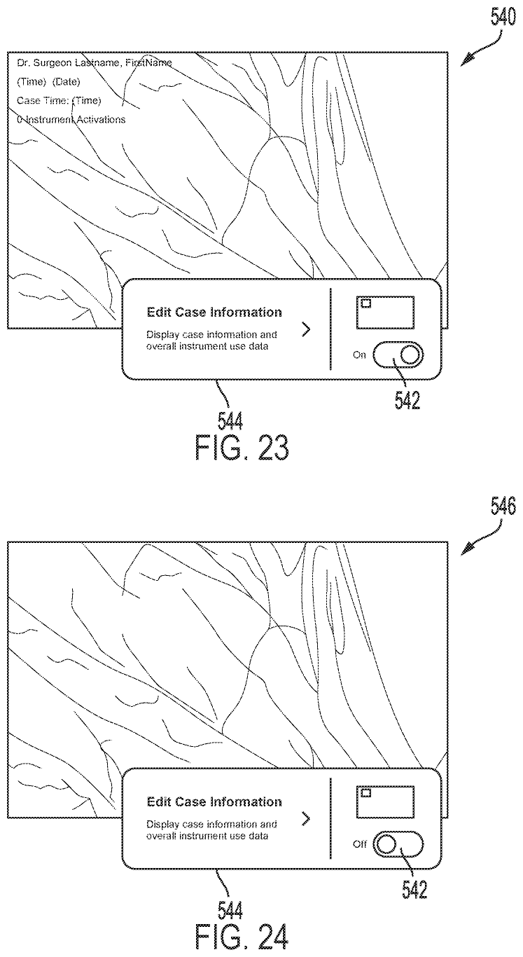

FIGS. 23-30 show behavior, customize overlay panels and modals. FIG. 23 illustrates a screen 540 with a top level switch 542 and an edit information screen 544 according to at least one aspect of the present disclosure. Tapping on the top level switch 542 hides/shows the panel on PSD. When the top level switch 542 is switched off as shown by the screen 546 in FIG. 24, the graphic changes to an empty state and opening the customization panel is disabled. In one example, the notification transition, may include a 0.3 sec transition, ease in and out and applies to both switch and graphic.

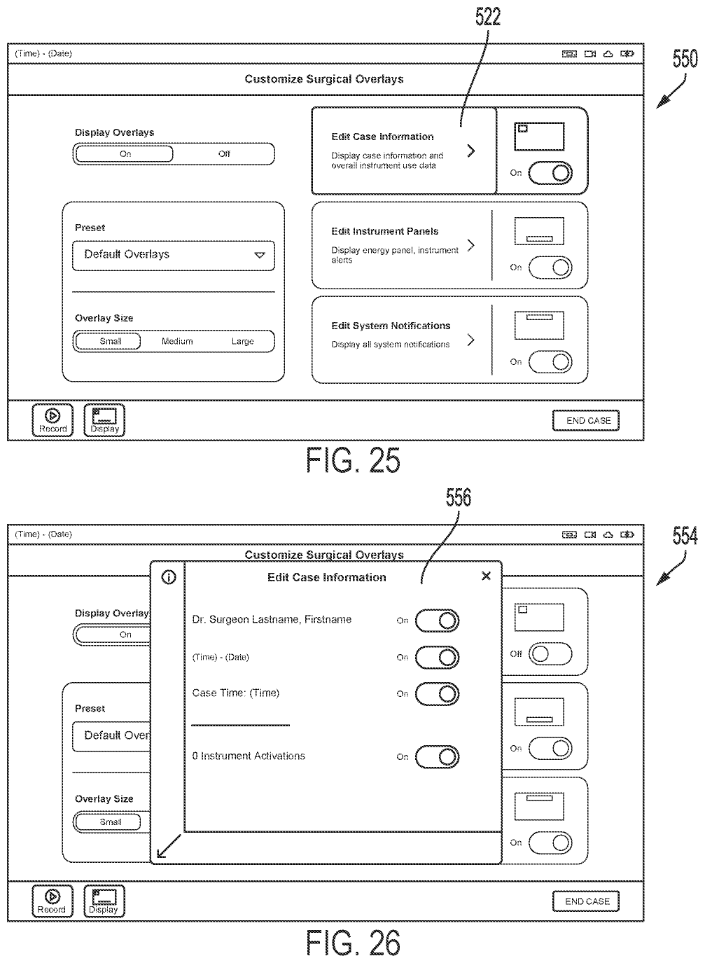

FIG. 25 illustrates a customized edit panel 550, which is opened by tapping on the text of the edit information screen 544 shown in FIG. 23 according to at least one aspect of the present disclosure. In some aspects, the background appears to dim. For example, the Edit Case Information panel 552 can remain normal with the other panels becoming dim. A user tapping on the Edit Case Information panel 552 opens the Edit case Information subpanel 556 in screen 554 as shown in FIG. 26. Notification transition includes a 0.3 sec transition, ease in and out for all aspects. The opacity may change from 0-100%. The scale may change from 0.8× to 1×, or any suitable scale. The X-position may shift a predetermined number of pixels from the open state, for example, −120 pixels. The Y-position may shift as well by a predetermined number of pixels. For example, Case info Starts: 194//Ends: 110; Instrument Starts 244//Ends: 110; and System Notification Starts: 381//Ends: 164.

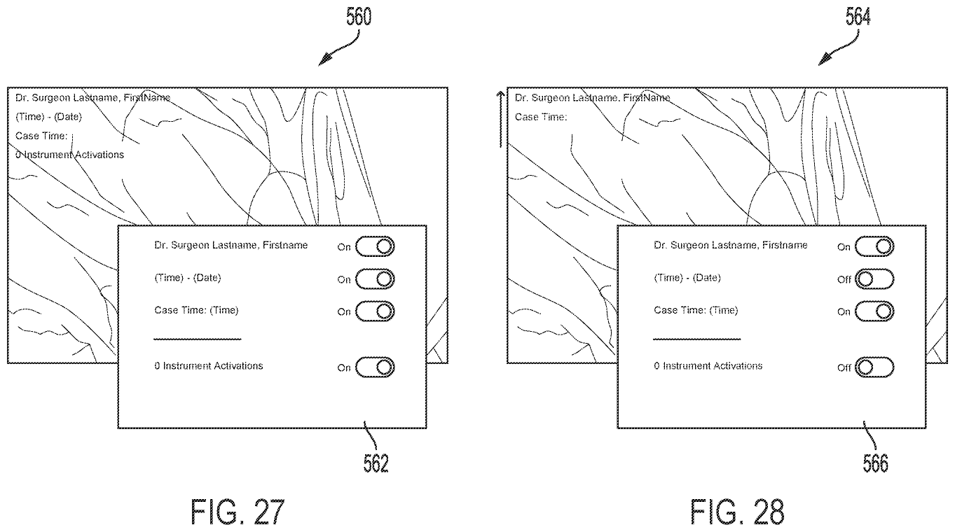

FIG. 27 illustrates a screen 560 with a customizable panel 562 according to at least one aspect of the present disclosure. This feature can be used to hide/show specific aspects on PSD panels. A user can tap on the X or outside of panel area to close. In one aspect, the screen 560 is displayed on a touch screen allowing a user to touch the screen to interact with the customizable panel 562. In an alternative aspect, a user can use a separate input device, e.g. a computer mouse, computer keyboard, etc., to make selections on the customizable panel 562. The screen 564 shown in FIG. 28 illustrates customized panel 566 where the content displayed on the screen 564 dynamically collapses based on the customized selection. In at least one aspect, the customized selection is specific to a surgeon. For example, each surgeon can have a preference on the data shown in the overlay for a type of surgical procedure. This overlay configuration could be stored in a database, as described in regard to FIGS. 16 and 17, for later use by the surgeon.

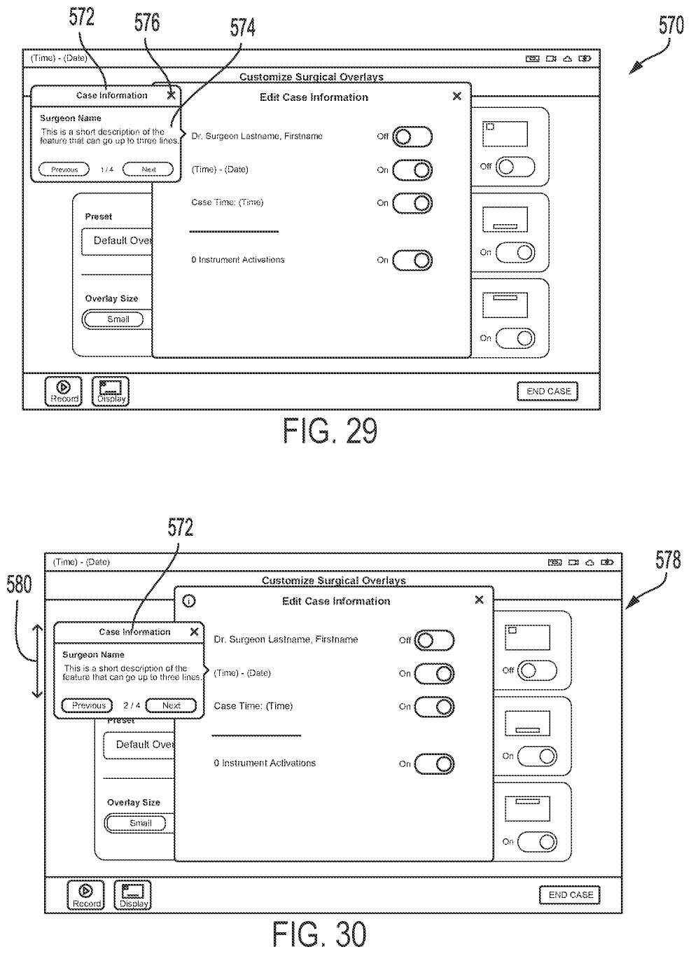

FIG. 29 illustrates an information panel screen 570 according to at least one aspect of the present disclosure. A user tapping on the information icon activates the information popup 572, which describes each line item. A user tapping next 574 will move the popup to the next item. A user can tap on the X 576 or outside of panel area closes the information popup 572. In one aspect, the information panel screen 570 can be displayed on a touch screen allowing a user to touch the screen to interact with the panel screen 570. In an alternative aspect, a user can use a separate input device, e.g. a computer mouse, computer keyboard, etc., to make selections on the panel screen 570. FIG. 30 shows the information popup 572 moved along an edge 580 of the screen 578 to the next item.

### Guided Troubleshooting System Connections

In order to take information from one system and overlay it on another, many connections across capital equipment need to be made. Sometimes, it is hard to identify which connections need to be made or it is unclear how to make the connections between equipment in the Operating Room (OR). The following techniques will help users troubleshoot connections more quickly, easily, and correctly.

This techniques described herein below are designed to guide users through the steps of troubleshooting various connections between capital equipment. For example, a connection or multiple connections may be disconnected simultaneously. It can be beneficial to a user if only relevant troubleshooting steps are shown. The system, e.g. a general purpose computer, surgical hub 6, or surgical hub 56, can determine whether cables are disconnected from the system itself—or—the system can determine when cables are plugged into the system but not connected to the other capital equipment by pinging. When multiple connections need to be made, a priority list will be used to determine which troubleshooting step will be shown first along with the order of any following trouble shooting steps. In at least one aspect, a general purpose computer, e.g. surgical hub 6 or surgical hub 56, generates the priority list to provide appropriate trouble shooting steps in a beneficial order to a user. In at least one aspect, the general purpose computer displays details of the trouble shooting steps to a user on a display, e.g. display 7, 9, 19, 35, 62, 65, 66, 67, and/or 89.

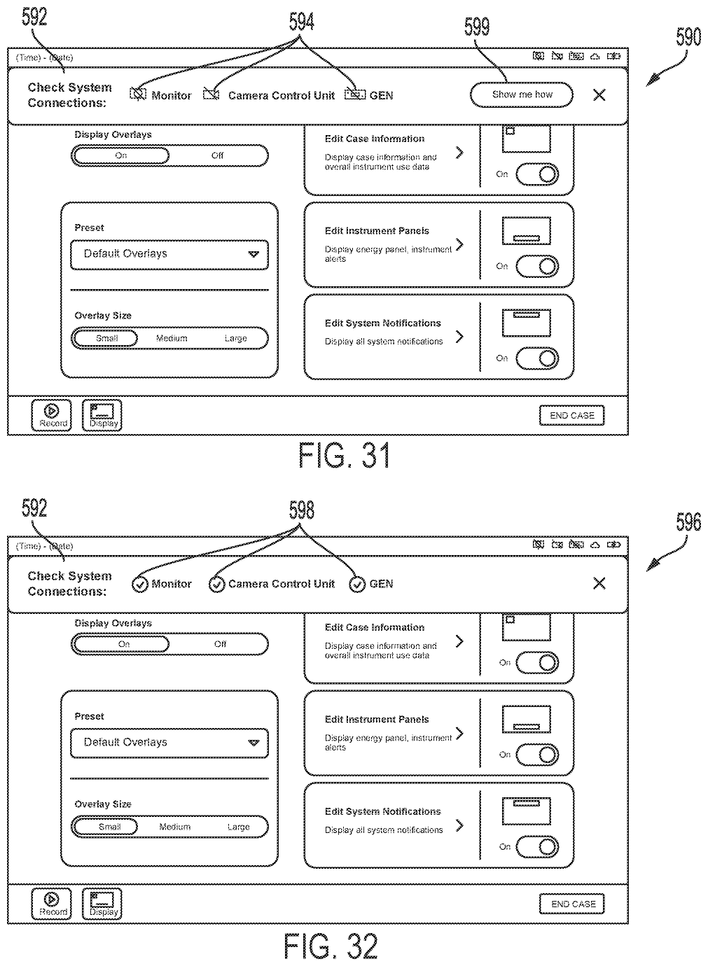

FIG. 31 illustrates a screen 590 with an overlaid system connection banner 592, which provides an overview of the connection status according to at least one aspect of the present disclosure as summarized below. In at least one aspect, a general purpose computer, e.g. surgical hub 6 or surgical hub 56, determines the devices that need connected for a surgical procedure and detects devices that are not currently connected. For example, the overlaid system connection banner 592 displays detected missing connections. A grayed icon 594, or slashed icon, indicates a missing connection. A checkmark icon 598 as shown in screen 596 in FIG. 32 indicates a successful connection. A “Show me how” button 599 shown in FIG. 31 disappears after all systems are connected as shown in FIG. 32. In one example, the overlaid system connection banner 592 may employ the general purpose computer icons.

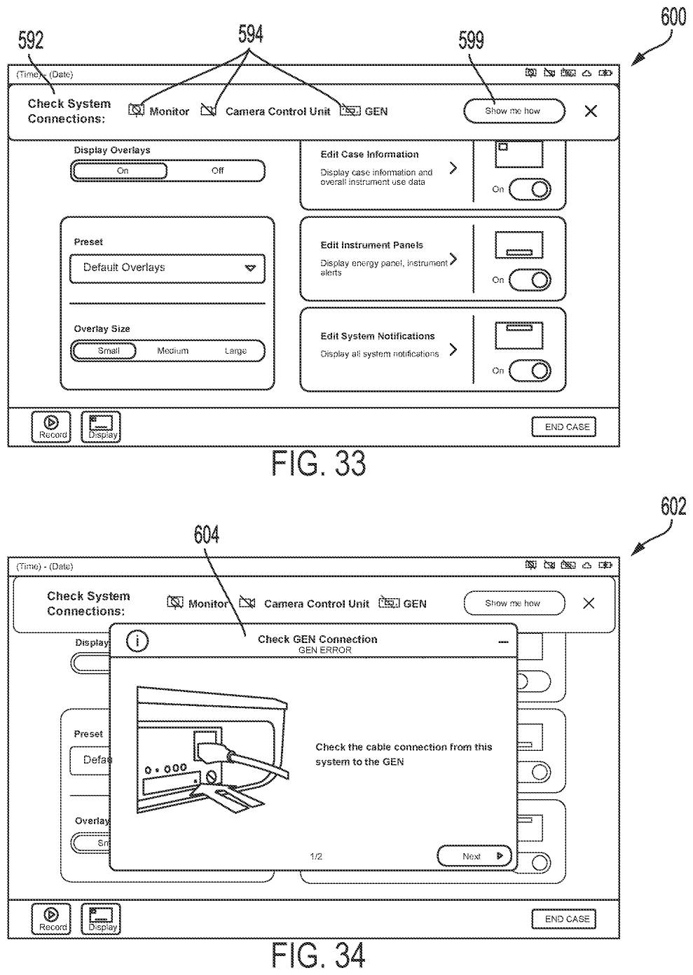

FIG. 33 illustrates a troubleshooting window 600 according to at least one aspect of the present disclosure. The troubleshooting window display shown in FIG. 34 displays a more detailed troubleshooting instruction in a pop-up window 604 shown in screen 602 that overlays (partially or completely) the primary interface. Tapping the “Show me how” button 599 on the banner 592, shown in FIG. 33, will open the larger pop-up window 604 with more detailed instructions. The order and number of steps is dependent on what is connected. Troubleshooting instructions can be communicated to a user through one of the following methods or combination of methods: Written description, Static Image, GIF, Video, and/or Voice. For example, one of the methods can be displayed or played on a general purpose computer to provide a user with a trouble shooting instruction.

As soon as the connection is resolved, the troubleshooting window 600 should disappear or advance to the next relevant troubleshooting step. When all connections are successfully made, the troubleshooting window 600 will close and return to the user interface.

In order to take information from one system and overlay it on another, many connections across capital equipment need to be made. Sometimes, it may be difficult to identify which connections need to be made or it's unclear how to make the connections between equipment in the OR.

Many connections need to be made to enable the overlay of instrument information on the primary surgical display (laparoscopic/endoscopic monitor), e.g. display 7, 9, 19, 35, 62, 66, 67, and/or 89. This workflow is designed to help guide users through the steps to troubleshoot the connections.

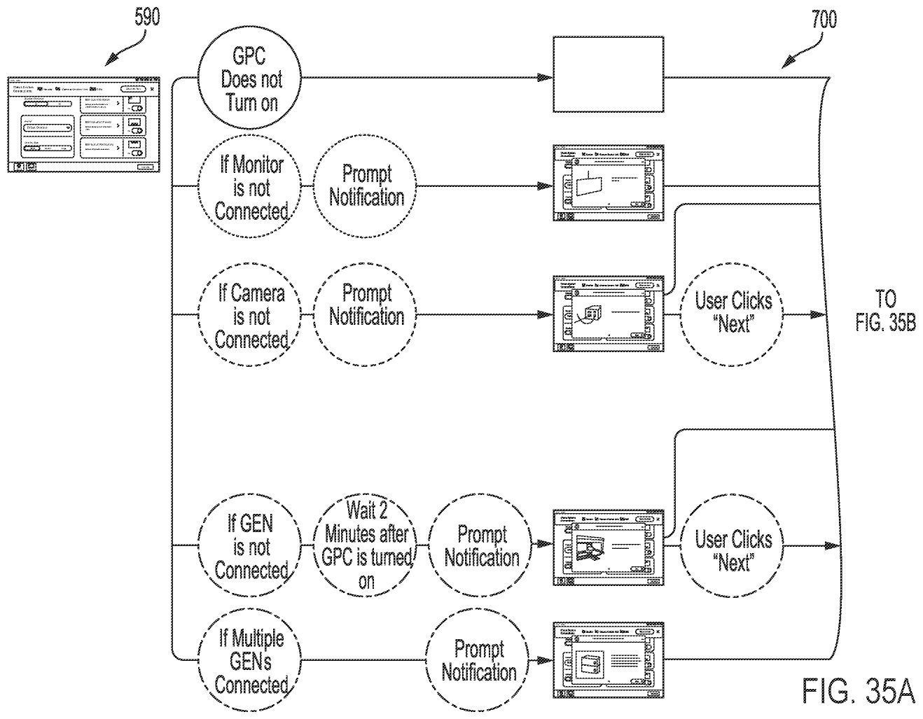

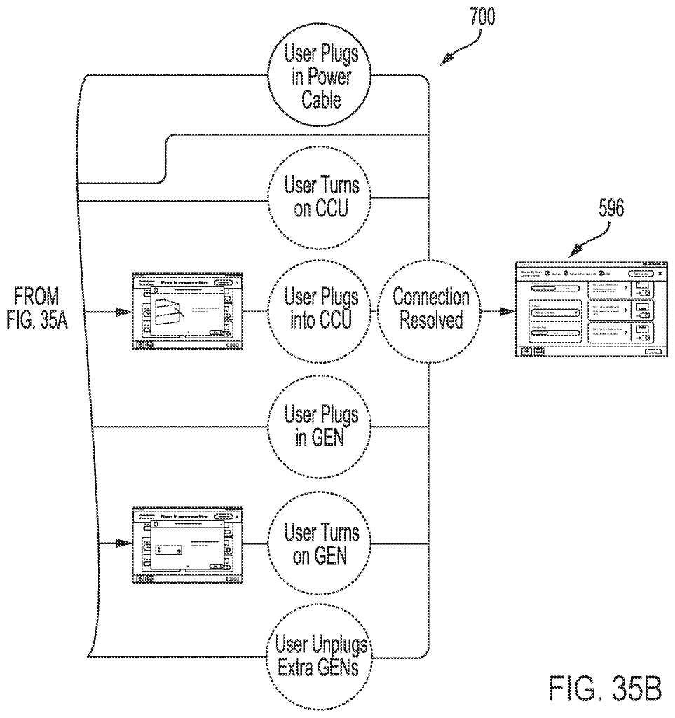

FIGS. 35A and 35B illustrate a troubleshooting flow diagram 700 that instructs a user on how to resolve connections, according to at least one aspect of the present disclosure. On the left (FIG. 35A) is the troubleshooting window 590 shown in FIG. 31 and on the right (FIG. is screen 596 shown in FIG. 32 where the connections are made. The troubleshooting flow diagram 700 illustrates different steps that a user can perform or that a GPC performs.

As shown in the troubleshooting flow diagram 700, if the GPC does not turn on, then a display of the GPC remains blank. The user can notice the blank display and then plug in the power cable of the GPC to resolve the connection.

As shown in the troubleshooting flow diagram 700, if a surgical monitor is not connected, the GPC detects that the surgical monitor is not connected and automatically provides a trouble shooting notification to a user. A display illustrates the trouble shooting notification of the surgical monitor not being connected to the user. The user can then proceed without that surgical monitor or perform steps to connect the surgical monitor.

As shown in the troubleshooting flow diagram 700, if the camera control unit (CCU) is not connected, then the GPC detects that the CCU is not connected and automatically provides a trouble shooting notification to the user on a display. The trouble shooting notification instructs the user to turn on the CCU. If the connection is not resolved, the user selects “next” and the GPC provides a second trouble shooting notification on the display. The second trouble shooting notification instructs the user to plug in the CCU. The user plugs in the CCU to resolve the connection. If the connection is resolved, then the GPC automatically removes the trouble shooting notification.

As shown in the troubleshooting flow diagram 700, if the generator (GEN) is not connected, then the GPC detects that the GEN is not connected and automatically provides a trouble shooting notification to the user on the display. If the GPC was just turned on, then the GPC will wait until 2 minutes after the GPC is turned on before sending the trouble shooting notification to the user. The trouble shooting notification instructs the user to plug in the GEN. The user then plugs in the GEN to resolve the connection. If the connection is not resolved, then the user selects “next” and the GPC provides a second trouble shooting notification to the display. The second trouble shooting notification instructs the user to turn on the GEN. The user then turns on the GEN to resolve the connection. If the connection is resolved, then the GPC automatically removes the trouble shooting notification.

As shown in the troubleshooting flow diagram 700, if multiple GENs are connected, then the GPC detects that multiple GENs are connected and automatically provides a trouble shooting notification to the user on the display. The trouble shooting notification instructs the user to unplug the extra GENs. The user then unplugs the extra GENs to resolve the connection. If the connection is resolved, then the GPC automatically removes the trouble shooting notification.

Those skilled in the art will recognize that, in general, tapping on a display or panel refers to making a selection on the display or panel. For example, an information panel can be displayed on a touch screen and touching the display makes the selection. As another example, selections on the information panel can be made by using a separate input device, e.g. a computer mouse, computer keyboard, etc.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of this disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, logic diagrams, and/or examples. Insofar as such block diagrams, logic diagrams, and/or examples contain one or more functions and/or operations, it will be understood by those within the art that each function and/or operation within such block diagrams, logic diagrams, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a control circuit, computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December, 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

The terms “proximal” and “distal” are used herein with reference to a clinician manipulating the handle portion of the surgical instrument. The term “proximal” refers to the portion closest to the clinician and the term “distal” refers to the portion located away from the clinician. It will be further appreciated that, for convenience and clarity, spatial terms such as “vertical”, “horizontal”, “up”, and “down” may be used herein with respect to the drawings. However, surgical instruments are used in many orientations and positions, and these terms are not intended to be limiting and/or absolute.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A computer implemented surgical system for use during a surgical procedure, the computer implemented surgical system comprising:
a near field communication (NFC) reader; and
a control circuit comprising a processor and a memory, wherein the memory stores instructions that, when executed by the processor, cause the control circuit to:
receive surgeon identification data indicative of a surgeon performing the surgical procedure;
receive a request to wirelessly pair with a surgical device based on the surgical device powering on;
receive device identification data indicative of the surgical device;
wirelessly pair the surgical device to the computer implemented surgical system based on the device identification data;
retrieve surgeon preferences relating to the paired surgical device and the surgeon from a database comprising a plurality of surgeon preferences for a plurality of surgical devices, wherein the surgeon preferences comprise an overlay configuration for at least one overlay relating to the surgical device; and
display the at least one overlay based on the overlay configuration on a primary surgical display, wherein the overlay is displayed overtop of information on the primary surgical display during the surgical procedure.

2. The computer implemented surgical system of claim 1, wherein the surgeon preferences further comprises control parameters of the paired surgical device, and wherein the memory stores further instructions that, when executed by the processor, cause the control circuit to transmit the control parameters to the surgical device.

3. The computer implemented surgical system of claim 1, wherein the at least one overlay relates to data received from the surgical device during the surgical procedure, and wherein the at least one overlay is configured to provide a warning to the surgeon based on the data exceeding a predetermined threshold.

4. The computer implemented surgical system of claim 1, wherein receive device identification data indicative of the surgical device comprises receiving the device identification data from a NFC card located proximal to the NFC reader, and wherein the NFC card is stored within the surgical device prior to the surgical procedure.

5. The computer implemented surgical system of claim 4, wherein the memory stores further instructions that, when executed by the processor, cause the control circuit to transmit, in response to receiving the request, instructions for wirelessly pairing the surgical device to the computer implemented surgical system, wherein the instructions are transmitted to the display, and wherein the instructions comprise requesting that the NFC card be placed proximal to the NFC reader.

6. The computer implemented surgical system of claim 5, wherein the memory stores further instructions that, when executed by the processor, causes the control circuit to provide feedback in response to the instructions being transmitted, wherein the feedback is configured to notify a user that a user input is required.

7. A computer implemented surgical system to automatically prompt for pairing of wireless devices during a surgical procedure, the computer implemented surgical system comprising:
a near field communication (NFC) reader; and
a control circuit comprising a processor and a memory, wherein the memory stores instructions that, when executed by the processor, cause the control circuit to:
receive first identification data indicative of a surgical device from a NFC card located proximal to the NFC reader;
compare the first identification data to a database comprising data of a plurality of surgical devices;
determine the surgical device of the plurality of surgical devices based on the comparison;
wirelessly pair with the surgical device to the computer implemented surgical system based on the data and the surgical device powering on; and
transmit confirmation of the pairing to a display.

8. The computer implemented surgical system of claim 7, wherein the memory stores further instructions that, when executed by the processor, cause the control circuit to:
receive second identification data indicative of a surgeon performing the surgical procedure;
retrieve, in response to receiving the second identification data, surgeon preferences relating to the surgical device from a database comprising a plurality of surgeon preferences for surgical devices; and
transmit the surgeon preferences to the surgical device.

9. The computer implemented surgical system of claim 8, wherein the surgeon preferences comprise an overlay configuration for at least one overlay relating to data received from the surgical device during the surgical procedure, and wherein the memory stores further instructions that, when executed by the processor, cause the control circuit to:
determine an overlay configuration based on the surgeon preferences; and
display the at least one overlay based on the overlay configuration on a primary surgical display, wherein the overlay is displayed overtop of information on the primary surgical display.

10. The computer implemented surgical system of claim 7, wherein the data indicative of the surgical device comprises a media access control (MAC) address, and wherein the database of a plurality of surgical devices comprises MAC addresses for a plurality of surgical devices.

11. The computer implemented surgical system of claim 7, wherein the NFC card is stored within the surgical device prior to the surgical procedure.

12. A computer implemented surgical system for use during a surgical procedure, the computer implemented surgical system comprising:
a control circuit comprising a processor and a memory, wherein the memory stores instructions that, when executed by the processor, cause the control circuit to:
receive an overlay configuration for at least one overlay from a secondary user interface, wherein the at least one overlay comprises at least one overlay element, and wherein the overlay configuration is based on preferences of a surgeon performing the surgical procedure; and
display the at least one overlay based on the overlay configuration on a primary surgical display, wherein the overlay provides information to the surgeon, and wherein the overlay is displayed overtop of information on the primary surgical display.

13. The computer implemented surgical system of claim 12, wherein the secondary user interface selects the overlay configuration from a database comprising a plurality of overlay configurations, and wherein the selected overlay configuration is based on the surgeon.

14. The computer implemented surgical system of claim 12, wherein the secondary user interface allows a user to control how the at least one overlay is displayed by adjusting position, scale, and amount of information shown for each overlay element of the at least one overlay.

15. The computer implemented surgical system of claim 14, wherein the secondary user interface allows the user to update an overlay configuration and store the updated overlay configuration in a database comprising a plurality of overlay configurations.

16. The computer implemented surgical system of claim 12, wherein the memory stores further instructions that, when executed by the processor, cause the control circuit to:
receive a second overlay configuration from the secondary user interface; and
adjust simultaneously all overlay elements of the at least one overlay based on the second overlay configuration.

17. The computer implemented surgical system of claim 12, wherein the at least one overlay relates to a surgical instrument, and wherein the memory stores further instructions that, when executed by the processor, cause the control circuit to:
receive real-time video data of a surgical site from a camera;
determine that the surgical instrument is not currently in a field of view of the camera; and
adjust, automatically, the at least one overlay based on the surgical instrument leaving the field of view, wherein the adjustment minimizes the at least one overlay.

18. The computer implemented surgical system of claim 17, wherein minimizing the at least one overlay comprises reducing a size of the overlay, removing the overlay, or changing an opacity of the overlay, or combinations thereof.

19. The computer implemented surgical system of claim 12, wherein the at least one overlay relates to a surgical instrument, and wherein the memory stores further instructions that, when executed by the processor, cause the control circuit to:
receive data indicative of interactions of the surgical instrument;
determine that the surgical instrument is inactive based on the data; and
adjust, automatically, the at least one overlay based on the surgical instrument remaining inactive for a threshold period of time, wherein the adjustment minimizes the at least one overlay.

20. The computer implemented surgical system of claim 12, wherein the secondary user interface is located outside of a sterile field, and wherein the surgical procedure is being performed within the sterile field.
